(19) United States
(12) Patent Application Publication
(10) Pub. No.: US 2025/0140404 A1
(43) Pub. Date: May 1, 2025

(54) GENERATION OF SYNTHETIC DOCTOR-PATIENT CONVERSATIONS

(71) Applicant: Oracle International Corporation, Redwood Shores, CA (US)

(72) Inventors: Karan Dua, Najibadad (IN); Praneet Pabolu, Bangalore (IN); Ranjeet Kumar Gupta, Bengaluru (IN)

(73) Assignee: Oracle International Corporation, Redwood Shores, CA (US)

(21) Appl. No.: 18/495,966

(22) Filed: Oct. 27, 2023

(51) Int. Cl. G16H 50/20; G06F 40/169; G06F 40/295; G16H 70/60

(52) U.S. Cl. CPC G16H 50/20; G06F 40/169; G06F 40/295; G16H 70/60

(57) ABSTRACT

Knowledge graph guide and entity controlled techniques for generating synthetic doctor-patient conversations. In one particular aspect, a method is provided that includes obtaining an original dataset containing textual dialogue associated with a plurality of individual doctor-patient conversations for training a machine learning model, constructing input data by using named entity recognition to capture and categorize named medical entities present in the dialogue, generating prepared input data by arranging the input data in an annotated turn-by-turn conversation format using an input data preparation algorithm having various control parameters, training the machine learning model using the prepared input data, utilizing a knowledge graph to identify a plurality of symptoms mapped to a randomly selected disease, and causing the trained machine learning model to generate a synthetic doctor-patient conversation by inputting the plurality of symptoms to the machine learning model as a first control parameter of a conversation generation control algorithm.

### FIELD

The present disclosure relates generally to artificial intelligence techniques, and more particularly, to techniques for generating synthetic doctor-patient conversations using medical knowledge graphs and/or doctor-patient conversation summaries to guide and control the synthetic doctor-patient conversation generation process.

### BACKGROUND

Artificial intelligence (AI) may be utilized in the medical field. For example, AI systems can be useful in identifying disease states to assist with diagnosing patients by analyzing images of tissue or cells. It may also be desirable to use AI systems and techniques to analyze doctor-patient conversations, where patients may describe symptoms, identify past activities, behaviors, or food and medicine intake, and provide personal information such as known medical conditions, drug allergies, family histories, etc. Theoretically, machine learning models can be trained on information provided during doctor-patient conversations and may be subsequently applied to future conversations to diagnose patient conditions, determine drug effect or interaction issues, predict risk, and for other purposes. The application of AI to doctor-patient conversations can potentially also help doctors better communicate with patients, such as by performing real-time analysis of patient-provided information and providing predictive results that can help to guide the doctor-patient conversation and improve a doctor's ability to make an accurate and timely diagnosis or to offer effective advice regarding treatment or prevention.

To properly train a machine learning model to perform tasks such as those described above based on doctor-patient conversation analysis, large amounts of training data are needed. Unfortunately, due to patient confidentiality, regulatory, and other reasons, only a very limited amount of such training data is publicly available and the publicly available datasets are either small, come from a specific domain, and/or involve significant access and licensing constraints relative to commercial usage. Human-generated doctor-patient conversation data may be used for model training, but it is expensive and time-consuming to collect. The use of manually curated doctor-patient conversation data may also be possible, but such data is often not representative of real-world conversations and is typically domain limited.

### BRIEF SUMMARY

Techniques disclosed herein relate generally to artificial intelligence techniques. More specifically and without limitation, techniques disclosed herein relate to the generation of synthetic doctor-patient conversations. Since the doctor-patient conversation data is generated (synthetic), patient privacy, data access, licensing, and other potential problems typically associated with the use of actual doctor-patient conversation data are obviated. The synthetic conversation data can be labeled and used to train downstream machine learning models such as, for example, entity extraction, automatic speech recognition, summarization, and PII/PHI detection models from unstructured dialogues.

Generating natural and engaging conversations is nonetheless a challenging task, as obstacles such as the need for large amounts of training data, the difficulty of capturing the nuances of human conversation, and the need to generate conversations that pertain to the topic of discussion by handling the inter-connections between medical entities and their associated symptoms, make generation process difficult. To overcome the above-identified difficulties, one version of a synthetic doctor-patient conversation generation technique may utilize data modeling based on knowledge graph guided named entity recognition (NER) and may employ known datasets and pre-trained models. According to this technique, a chosen model may initially be finetuned using multiple data preprocessing steps across multiple folds/stages and then mappings derived from medical knowledge graphs can be used to guide turn-by-turn generation of doctor-patient dialogues. Another version of a synthetic doctor-patient conversation generation technique may utilize existing doctor-patient conversation summaries as input. This technique can utilize multi-stage, turn-by-turn, control parameter based finetuning for domain alignment and improved conversation generation diversity. Data augmentation and filler word redistribution strategies may also be employed to further improve synthetic doctor-patient conversation generation diversity. Downstream machine learning models can be trained on labeled synthetic conversation data generated by the knowledge graph guided conversation generation technique, on synthetic conversation data generated by the conversation summaries guided conversation generation technique, or both. For example, synthetic doctor-patient conversation data generated by a synthetic doctor-patient conversation generation technique described herein can be used to train a model to identify and redact personal health information (PHI) or personal identifiable information (PII) from text, to recreate doctor-patient conversations from summaries such as clinician SOAP notes, or for enhancement of model automatic speech recognition (ASR) performance.

In various embodiments, a computer-implemented method is provided that includes: obtaining an original dataset for training a machine learning model to generate a synthetic doctor-patient conversation, the original dataset including data in the form of textual dialogue associated with each of a plurality of individual doctor-patient conversations; constructing input data by performing a named entity recognition operation on the dialogue of the original dataset that captures and categorizes named medical entities present in the dialogue; generating prepared input data by arranging the input data in an annotated turn-by-turn conversation format using an input data preparation algorithm having various control parameters; training the machine learning model using the prepared input data; randomly selecting a disease from a medical knowledge graph and capturing a plurality of symptoms that are mapped to the selected disease; and causing the trained machine learning model to generate a turn-by-turn synthetic doctor-patient conversation according to a conversation generation control algorithm having various control parameters, by inputting the plurality of symptoms to the machine learning model as a first control parameter of the conversation generation control algorithm and randomly assigning a first speaker.

In some embodiments, the named medical entities are selected from the group consisting of biomedical information, personal identifying information, personal health information, and combinations thereof.

In some embodiments, constructing the input data further comprises post-processing the named medical entities to remove any tagging anomalies resulting from the named entity recognition operation; and performing an additional medical named entity recognition operation on the post-processed named medical entities using at least one model trained on a biomedical corpus.

In some embodiments, the control parameters of the input data preparation algorithm and the conversation generation control algorithm include symptoms/topics, entities sampled from the symptoms/topics, speaker identity, conversation turn length, remaining conversation turns, and context; and a value of the context control parameter for a given conversation turn comprises the dialogue of all preceding conversation turns, when there is at least one preceding conversation turn.

In some embodiments, the format of the prepared input data is:

[TOPICS] <symptom values> [ENTITIES] <entity values> [SPEAKER]
<doctor/patient> [TURN_LENGTH]
<length of the turn>[REMAINING_TURNS]
<number of turns left> [CONTEXT] <previous dialogues if available>

In some embodiments, training the machine learning model includes multistage finetuning of the machine learning model, and the multistage finetuning comprises: first-fold finetuning using a medical question answering dataset; following the first-fold finetuning, second-fold finetuning guided by the input data preparation algorithm and the control parameters thereof and using a dataset including data in the form of textual dialogue associated with each of a plurality of individual non-medical specific conversations; and following the second-fold finetuning, final-fold finetuning guided by the input data preparation algorithm and the control parameters thereof and using a dataset including data in the form of textual dialogue associated with each of a plurality of individual medical-specific conversations, wherein the symptoms/topics control parameter of the input data preparation algorithm is composed of keywords previously derived from a dataset including data in the form of textual dialogue associated with each of a plurality of individual non-medical conversations.

In some embodiments, performance of the trained machine learning model is evaluated by analyzing the generated turn-by-turn synthetic doctor-patient conversation using a metric selected from the group consisting of recall-oriented understudy for gisting evaluation, n-gram diversity score, unique n-gram count, and combinations thereof.

In various embodiments, a system is provided that includes one or more data processors and a non-transitory computer readable storage medium containing instructions which, when executed on the one or more data processors, cause the one or more data processors to perform part or all of one or more methods disclosed herein.

In various embodiments, a computer-program product is provided that is tangibly embodied in a non-transitory machine-readable storage medium and that includes instructions configured to cause one or more data processors to perform part or all of one or more methods disclosed herein.

The techniques described above and below may be implemented in a number of ways and in a number of contexts. Several example implementations and contexts are provided with reference to the following figures, as described below in more detail. However, the following implementations and contexts are but a few of many.

### BRIEF DESCRIPTION OF THE DRAWINGS

FIG. 1 illustrates a process flow for a training a machine learning model to perform knowledge graph guided and entity controlled synthetic doctor-patient conversation generation.

FIG. 2 illustrates a process flow for preparing machine learning model input data by implementing a turn-by-turn input data preparation algorithm.

FIG. 3 is one example of medical knowledge graph.

FIG. 4 is one example of a combined medical knowledge graph that includes the medical knowledge graph of FIG. 3 overlaid on a separate precision medicine multi-modal knowledge graph.

FIGS. 5A-5B illustrate a process flow for the machine learning model execution of a knowledge graph guided and entity controlled synthetic doctor-patient conversation generation control algorithm to generate a multiple-turn synthetic doctor-patient conversation.

FIGS. 6A-6D present one example of a synthetic doctor-patient conversation generated by a trained knowledge graph guided and entity controlled synthetic doctor-patient conversation generation machine learning model.

FIG. 7A illustrates a multistage finetuning portion of a process flow for training a machine learning model to perform conversation summary guided synthetic doctor-patient conversation generation.

FIG. 7B illustrates a process flow for performing final-stage finetuning on a conversation summary guided synthetic doctor-patient conversation generation machine learning model.

FIG. 8 is a table presenting various information associated with a filler word redistribution operation of a conversation summary guided synthetic doctor-patient conversation generation machine learning model final-stage finetuning process.

FIG. 9 is a table presenting various information associated with a frequent word replacement operation of a conversation summary guided synthetic doctor-patient conversation generation machine learning model final-stage finetuning process.

FIG. 10 illustrates a process flow for inference of a trained conversation summary guided synthetic doctor-patient conversation generation machine learning model.

FIG. 11 illustrates a process flow for training a machine learning model to perform knowledge graph guided synthetic doctor-patient conversation generation.

FIG. 12 is a block diagram illustrating one pattern for implementing a cloud infrastructure as a service system, according to at least one embodiment.

FIG. 13 is a block diagram illustrating another pattern for implementing a cloud infrastructure as a service system, according to at least one embodiment.

FIG. 14 is a block diagram illustrating another pattern for implementing a cloud infrastructure as a service system, according to at least one embodiment.

FIG. 15 is a block diagram illustrating another pattern for implementing a cloud infrastructure as a service system, according to at least one embodiment.

FIG. 16 is a block diagram illustrating an example computer system, according to at least one embodiment.

### DETAILED DESCRIPTION

In the following description, for the purposes of explanation, specific details are set forth in order to provide a thorough understanding of certain embodiments. However, it will be apparent that various embodiments may be practiced without these specific details. The figures and description are not intended to be restrictive. The word “exemplary” is used herein to mean “serving as an example, instance, or illustration.” Any embodiment or design described herein as “exemplary” is not necessarily to be construed as preferred or advantageous over other embodiments or designs.

### Introduction

Artificial intelligence (AI) has many applications. For example, in a medical setting, AI can be applied to drug discovery, and can be used to diagnose disease, to predict disease, to provide a disease prognosis, to identify potential drug interactions, for medical imaging analysis, and for other purposes. AI may also be usable to improve doctor-patient communications, patient care, or the patient experience, such as by function as a physician's assistant or otherwise. Machine learning models can also be trained for entity extraction, automatic speech recognition, summarization, and personal identifiable information (PII) or protected health information (PHI) detection.

In order for a machine learning model to perform operations such as entity extraction, automatic speech recognition, summarization, or PII/PHI detection, the machine learning model must be able to recognize and understand dialogue, particularly doctor-patient conversations with respect to some desired model outputs. A machine learning model can be trained for this purpose, but training requires large amounts of doctor-patient conversation data and doctor-patient conversation data is not readily available in such large amounts due to patient privacy, data access, licensing, and other issues. Accordingly, a different approach is needed to address the challenges associated with training a machine learning model on doctor-patient conversation data.

In various embodiments, a computer-implemented method is provided that includes: a computer-implemented method is provided that includes: obtaining an original dataset for training a machine learning model to generate a synthetic doctor-patient conversation, the original dataset including data in the form of textual dialogue associated with each of a plurality of individual doctor-patient conversations; constructing input data by performing a named entity recognition operation on the dialogue of the original dataset that captures and categorizes named medical entities present in the dialogue; generating prepared input data by arranging the input data in an annotated turn-by-turn conversation format using an input data preparation algorithm having various control parameters; training the machine learning model using the prepared input data; randomly selecting a disease from a medical knowledge graph and capturing a plurality of symptoms that are mapped to the selected disease; and causing the trained machine learning model to generate a turn-by-turn synthetic doctor-patient conversation according to a conversation generation control algorithm having various control parameters, by inputting the plurality of symptoms to the machine learning model as a first control parameter of the conversation generation control algorithm and randomly assigning a first speaker.

In some instances, training the machine learning model includes multistage finetuning of the machine learning model, and the multistage finetuning comprises: first-fold finetuning using a medical question answering dataset; following the first-fold finetuning, second-fold finetuning guided by the input data preparation algorithm and the control parameters thereof and using a dataset including data in the form of textual dialogue associated with each of a plurality of individual non-medical specific conversations; and following the second-fold finetuning, final-fold finetuning guided by the input data preparation algorithm and the control parameters thereof and using a dataset including data in the form of textual dialogue associated with each of a plurality of individual medical-specific conversations, wherein the symptoms/topics control parameter of the input data preparation algorithm is composed of keywords previously derived from a dataset including data in the form of textual dialogue associated with each of a plurality of individual non-medical conversations.

### Synthetic Doctor-Patient Conversation Generation

In order to provide a sufficient volume of doctor-patient conversation data to properly train a machine learning model, the approaches described herein are operative to generate synthetic doctor-patient conversations that obviate the confidentiality concerns and other issues that limit the availability of and access to actual doctor-patient conversation data. One embodiment of a doctor-patient conversation generation technique may utilize data modeling based on knowledge graph guided named entity recognition (NER) and may employ known datasets and pre-trained models. According to another embodiment, a synthetic doctor-patient conversation generation technique may utilize existing doctor-patient conversation summaries as input. A synthetic doctor-patient conversation generation model can be trained to generate synthetic doctor-patient conversations based on one or both of these techniques, and downstream machine learning models can be trained on the synthetic doctor-patient conversations for purposes of performing various operations, such as one or more of the operations identified above.

Conversation data from known datasets or conversation data embodied in existing conversation summaries can be used to finetune a synthetic doctor-patient conversation generation model. In the case of knowledge graph guided synthetic doctor-patient conversation generation, seed data can also be generated for initiating conversation generation at model inference. The machine learning model training process can utilize a turn-by-turn conversation flow that is governed by a set of control parameters so that a trained synthetic doctor-patient conversation generation model will ultimately generate natural and engaging synthetic doctor-patient conversations such as those that typically occur during doctor-patient interactions.

### A. Knowledge Graph Guided and Entity Controlled Synthetic Doctor-Patient Conversation Generation

As described in more detail below, a knowledge graph is a data structure that represents knowledge as a network of interconnected entities and their relationships. Knowledge graphs are an effective way of organizing information and enable more efficient and accurate data analysis and decision-making. Relative to the training of a machine learning model, a knowledge graph can provide a structured representation of entity relationships (e.g., patient activity and symptom entities). As such, a knowledge graph can be used to guide a synthetic doctor-patient conversation generation process.

In a typical real-world conversation between a doctor and a patient regarding a medical issue, various important information is likely to be consistently discussed. This information may include personal information, such as the patient's age and sex, and a description of how the patient feels or has been feeling, such as may be described in the form of symptoms. A patient may also provide information about recent behavior, activities, food consumption, prescription or other drug use, and a myriad of other details that can help a physician diagnose a patient condition or otherwise assist/advise the patient. With respect to training a synthetic doctor-patient conversation generation machine learning model (also referred to hereinafter as “synthetic doctor-patient conversation generation machine learning model”), these pieces of information can serve as entities that can be classified into categories. Identified and classified entities can be used in combination with a knowledge graph to guide and control the generation of synthetic conversation data.

### A.1. Data Modeling

An ideal dataset for use in a synthetic doctor-patient conversation generation process may include conversation data having all major entities (e.g., PII/PHI and medical entities) annotated in each conversation turn of the dialogue, where a “turn” is defined simply as a given portion of a conversation where one person is speaking and another person(s) is listening. Thus, in a doctor-patient conversation, for example, a first conversation turn might be initial dialogue provided by the doctor, a second conversation turn would be a response or other dialogue provided by the patient, and so on. Relative to model training data, an overall conversation (doctor-patient or otherwise) may be followed in a turn-by-turn fashion, and entities may be identified and annotated in each turn. It is also possible during a conversation for the same speaker to have multiple continuous turns, such as when the speaker finishes a sentence and then begins a new sentence before another speaker speaks. In such a case, it is possible to group at least some of the multiple continuous turns together to create an alternating sequence of patient-doctor conversation turns in the training data.

As discussed above, publicly available doctor-patient conversation data is very limited, and is of insufficient volume to train a synthetic doctor-patient conversation generation model to have reasonable accuracy if using the available data alone. However, some curated doctor-patient conversation data is publicly available. For example, both the PriMock57 dataset and the Nature dataset are publicly available. The PriMock57 dataset contains textual dialogue associated with 57 simulated doctor-patient conversations that each average 97 conversation turns. The Nature dataset is also a simulated doctor-patient conversation dataset, which was assembled by a team of doctors and medical students and includes approximately 300 conversation (data points). For purposes of training a synthetic doctor-patient conversation generation model according to at least some embodiments of the present disclosure, the PriMock57 dataset can be combined with the Nature dataset and modeled in a manner that allows for associating entities with dialogues, as described in more detail below. In other embodiments, other sets of doctor-patient conversation data may be used individually or in combination and modeled in a manner that allows for associating entities with dialogues, as described in more detail below.

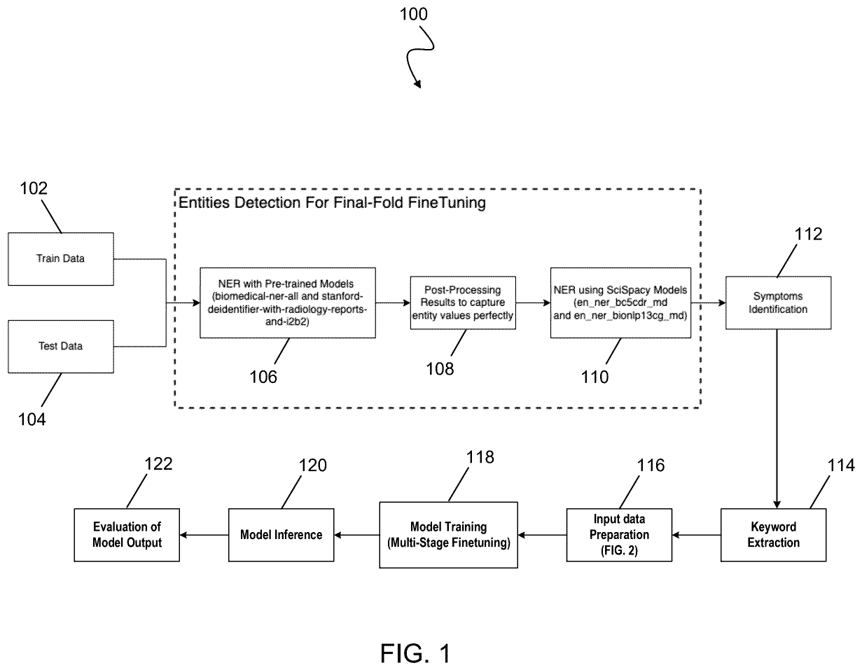

One embodiment of a knowledge graph guided synthetic doctor-patient conversation generation process 100 is represented generally in the flowchart of FIG. 1. As indicated, a dataset comprising data from one or more sets of doctor-patient conversation data such as curated data from the combined PriMock57 and Nature datasets may be split into a training dataset 102 and a test dataset 104. The training dataset 102 and the test dataset 104 may be provided to each of the three individual data modeling operations 106, 108, 110 of the collective data modeling operation. The three data modeling operations 106, 108, 110 cooperate to detect entities present in the simulated conversations contained in the training datasets 102, 104.

### A.1.1. Named Entity Recognition Using a Pre-Trained Model(s)

Simply stated, named entity recognition (NER) is a form of natural language processing (NLP) whereby key information (entities) in text can be identified and categorized. As a first step in the data modeling process associated with embodiments of the knowledge graph guided synthetic doctor-patient conversation generation process, a NER operation 106 can be applied to the combined data of the training dataset 102 and the test dataset 104. In one embodiment, NER is applied to the data using two pre-trained models, namely the biomedical-NER-all model and the Stanford AIMI/stanford deidentifier-with-radiology-reports-and-i2b2 model, which collectively provide for the identification and classification of biomedical, PHI and medical entities in text. In other embodiments, NER is applied to the data using one or more pre-trained NER models, which individually or collectively provide for the identification and classification of medical data including biomedical, PHI and medical entities in text. The NER operation 106 may be performed on the combined data using these pre-trained models in a turn-by-turn fashion. As the pre-trained models are able to detect entities with higher accuracy for smaller sentences versus larger sentences, the speaker identifications appearing in the simulated doctor-patient conversations of the combined data may be removed prior to performing the NER operation 106.

As represented in Table 1 below, which reflects a portion of one simulated doctor-patient conversation obtained from the combined data, the NER operation 106 using the pre-trained models is preferably designed to break down the dialogue into simpler, smaller sentences at a conversation turn level rather than at a whole conversation level. It may be observed that use of the combined pre-trained models also detects and captures several entities in the first turn having values such as “stool,” “normal,” “pearly,” and “35 years” that are present at some point in the dialogue. The NER process using the pre-trained models also categorizes the detected entities. The categories are referred to as “entity names” in Table 1, and include relative to the first conversation turn, “diagnostic procedure” associated with the “stool” entity, “lab value” associated with the “normal” entity, “detailed description” associated with the “pearly” entity, and “age” associated with the “35 years” entity. As indicated, the NER operation 106 can be repeated on the other conversation turns of the simulated conversation represented in Table 1, and can also be applied to the other simulated conversations provided by the combined data to identify/detect other entities of interest that appear in the simulated doctor-patient conversations.

TABLE 1

| Dialogue turn | entity names | entity values |
|---|---|---|
| Patient: In the morning, the stool is normal and | [‘Diagnostic_procedure’, | [‘stool’, ‘normal’, |
| then thin in the evening, there is a bit of pearly dry | ‘Lab_value’, | ‘pearly’, ‘35 |
| stool, what could be the case? (male, 35 years old) | ‘Detailed_description’, | years’] |
| | ‘Age’] | |
| Doctor: Hello, how long has this been the case? Is | [‘Sign_symptom’] | [‘cold’] |
| the diet regular? Have you eaten stimulating food | | |
| or caught a cold? | | |
| Patient: It has been four or five days. I had dry stool | [‘Sign_symptom’, | [‘dry,’ ‘stool’, |
| when I took amoxicillin for rhinitis before, and then | ‘Activity’, ‘Medication’, | ‘amoxicillin’, |
| I took laxative for a few days. Not it is like this, and | ‘Biological_attribute’, | ‘rhinitis’, ‘cold’] |
| everything else is normal. Have not eaten | ‘Sign_symptom’] | |
| stimulating food and caught a cold. | | |
| Doctor: How many times a day do you poop? | [‘Activity’] | [‘poop’] |
| Patient: Once a day is normal, sometimes at night, | [‘DATETIME’] | [‘night’] |
| sometimes not. | | |

### A.1.2. Post-Processing of Named Entity Recognition Results

It was discovered that using the pre-trained models to perform the NER process on the combined simulated conversation data can result in various entity tagging anomalies. Some of these anomalies are exemplified in Table 2 below, which reflects a portion of another simulated doctor-patient conversation obtained from the combined training dataset. For example, the pre-trained models may tag a patient's age as separate “29” and “years” entities rather than as a single “29 years” entity. In another example, hashtags may be present due to model tokenization. Consequently, model training embodiments may perform a second, post-processing operation 108, on the NER results to overcome these issues. Various examples of corrections resulting from such a post-processing operation 108 are represented in Table 2. For example, separately tagged “29” and “years” entities appearing in the first conversation turn have been combined into a single “29 years” entity that has been properly categorized as “age,” and separately tagged “don,” apostrophe, and “t” entities appearing in the second turn have been combined into a single “don't” entity that has been properly categorized as “sign_symptom.” Other post-processing corrections in other conversation turns may also be observed in Table 2, such as the removal of hashtags that have been inserted into and split up the word “palpitations” into several word fragments.

TABLE 2

| Dialogue Turn | Entities | Post Processing |
|---|---|---|
| Patient: Hello, there is a dull pain around the belly button, | (‘29’, ‘B-Age’, [105, 107]), | (‘29 years’, ‘Age’, [105, |
| I don't know what's going on (female, 29 years old). | (‘years’, ‘I-Age’, [108, 113]), | 113]), |
| Patient: Hello, there is a dull pain around the belly button, | (‘don’, ‘B-Sign_symptom’, | (‘don't’, ‘Sign_symptom’, |
| I don't know what's going on (female, 29 years old). | [69, 72]), | [69, 74]), |
| | (‘′’, ‘I-Sign_symptom’, [72, | |
| | 73]), | |
| | (‘t’, ‘I-Sign_symptom’, [73, | |
| | 74]), | |
| I get palpitations. | (‘pal’, ‘B-Sign_symptom’, [6, | (‘palpitations’, |
| | 9]), | ‘Sign_symptom’, [6, 18]) |
| | (‘##pit’, ‘I-Sign_symptom’, | |
| | [9, 12]), | |
| | (‘##ations’, ‘I- | |
| | Sign_symptom’, [12, 18]), | |
| Diarrhoea can be a reason. | (‘dia’, ‘B-Sign_symptom’, [0, | (‘Diarrhoea’, |
| | 3]), | ‘Sign_symptom’, [0, 8]) |
| | (‘##rr’, ‘I-Sign_symptom’, | |
| | [3, 5]), | |
| | (‘##hoea’, ‘I- | |
| | Sign_symptom’, [5, 8]) | |

### A.1.3. Medical Named Entity Recognition

The previously described pre-trained models such as the biomedical-ner-all and Stanford AIMI/stanford deidentifier-with-radiology-reports-and-i2b2 models are adept at generating a number (e.g., 20) entities from the combined training data. However, the pre-trained models lack the particular ability to detect many medical-specific entities such as various diseases, chemicals, pharmaceuticals, types of cancers, etc. As such, a third data modeling operation 110 according to this particular data model embodiment can be employed and may be directed to the detection/identification particularly of medical-specific terminology (entities) within the simulated conversations of the combined training data.

While synthetic doctor-patient conversation generation embodiments are not limited thereto, a particular embodiment described herein for purposes of illustration uses the SciSpacy model to perform a medical NER operation 110 on the combined training data. It should be understood that it may be possible to use other models capable of processing medical text such as BioBERT to perform the medical NER operation 110 on the combined training data. The SciSpacy model is built on top of the open source NLP library, Spacy. The SciSpacy model is specifically designed to process scientific, biomedical and clinical text data, and also provides pre-trained models, tokenizers and entity recognizers tailored for the biomedical domain. The SciSpacy model further offers a range of features and capabilities for biomedical text processing, and NER functionality for identifying entities like diseases, drugs, genes and more. These features can be useful at least in this embodiment, as the data modeling process relies on the use of entities captured from the publicly available combined training datasets to control the flow of synthetic doctor-patient conversation generation. Two SciSpacy models are utilized in this particular synthetic doctor-patient conversation generation embodiment. One of these SciSpacy models is the en_ner_bc5cdr_md model, which has been trained on the BC5CDR corpus to classify two entities. The second of these SciSpacy models is the en_ner_bio nlp13cg_md model, which has been trained on the BIONLP13CG corpus to classify 16 entities. As used herein, the en_ner_bc5cdr_md model and the en_ner_bio nlp13cg_md model operate to confidently identify and classify entities at each conversation turn.

In similar fashion to the first NER data modeling step, the medical NER operation 110 preferably uses the pre-trained models such as the SciSpacy models to break down the dialogue into simpler, smaller sentences at a conversation turn level and to detect medical-specific entities within the conversation turns. This may be observed in Table 3, below, which reflects a portion of another simulated doctor-patient conversation obtained from the combined training dataset. For example, it may be observed in Table 3 that the medical NER operation 110 detects several entities present in the dialogue of the first conversation turn such as “feverish,” “paracetamol,” and “tablets,” and detects the entity “lung cancer” that is present in the dialogue of the second conversation turn. The medical NER operation 110, using the pre-trained models, also categorizes the detected entities. In this case, the entities “feverish,” “paracetamol,” and “tablets” present in the first conversation turn are properly categorized as “disease,” “chemical,” and “simple chemical,” respectively. Likewise, the “lung cancer” entity that is present in the dialogue of the second conversation turn is properly categorized as “disease, cancer.” As indicated, the medical NER operation 110 can be repeated on the other conversation turns of the simulated conversation represented in Table 3. The medical NER operation 110 can also be applied to the other simulated conversations provided by the combined dataset to identify/detect other medical entities of interest that appear in the simulated doctor-patient conversations.

TABLE 3

| Dialogue turn | entity names | entity values |
|---|---|---|
| Doctor: If you are feeling feverish and weak, eh taking some | [‘DISEASE’, | [‘feverish’, |
| paracetamol, uh, two tablets up to four times a day for the | ‘CHEMICAL’, | ‘paracetamol’, |
| first few days can also help | ‘SIMPLE_CHEMICAL’] | ‘tablets’] |
| Patient: Think my grandpa died of lung cancer, actually he | [‘DISEASE’, | [‘lung cancer’] |
| was a pretty big smoker too though | ‘CANCER’] | |
| Patient: Yeah, it's uh, no it's pretty, it's pretty well, it seems | [‘CHEMICAL’, | [‘Tylenol’, |
| the same each time. It kind of lasts like 20 minutes or so, um | ‘CHEMICAL’, | ‘Advil’, ‘Alka- |
| and I've tried Tylenol and I've tried Advil um and I've even | ‘SIMPLE_CHEMICAL’] | Seltzer’] |
| tried like a like an Alka-Seltzer and it hasn't really helped very | | |
| much. | | |
| Doctor: Ok so aside from the eczema, it sounds like you're | [‘DISEASE’] | [‘eczema’] |
| pretty healthy, eh? | | |

### A.1.4. Symptom Determination

An additional symptoms determination (identification) data modeling operation 112 may be performed to mine the combined training dataset for entities in the form of patient symptoms. In this particular embodiment, symptom entities discovered within the combined training dataset are not used for further model training (e.g., finetuning), but instead will serve as seed data in a subsequent inference phase of the synthetic doctor-patient conversation generation model. For example, in order to initiate synthetic doctor-patient conversation generation by the trained synthetic doctor-patient conversation generation model, starter dialogue having a context based on patient symptoms can be used. Patient symptoms are usually associated with a disease or some other problem that a patient is experiencing. Consequently, a disease can be selected at random, and a pre-trained model such as the SciSpacy model can be used to capture as entities all the relevant symptoms that are associated with the selected disease. The captured symptoms can then be used downstream as starter dialogue to initiate synthetic doctor-patient conversation generation by the trained synthetic doctor-patient conversation generation model. Other starter dialogue may be generated and used to initiate synthetic doctor-patient conversation generation by the trained synthetic doctor-patient conversation generation model in other embodiments.

Although a disease may be randomly selected relative to creating starter dialogue for use in a downstream synthetic doctor-patient conversation generation model inference process, disease and symptom selection may be guided, nonetheless. For example, and without limitation, a knowledge graph may be consulted for its structured representation of diseases and associated symptoms prior to mining the combined curated training dataset for symptoms. The use of a pre-trained model such as the SciSpacy model to capture patient symptoms that can be used as conversation starter dialogue in a synthetic doctor-patient conversation generation model inference process is reflected by the disease and symptom terms appearing in Table 4 below. For example, it can be observed that in an instance where “bladder cancer” was randomly selected as the disease, the pre-trained model returned “pain,” “blood in urine,” “abdominal pain,” and a number of additional symptoms that can serve as seed data in a trained synthetic doctor-patient conversation generation model inference phase.

TABLE 4

| Disease | entity names |
|---|---|
| Abdominal Pain | Fever, Sandhoff Disease, Tiglic Acidemia, Spinocerebellar ataxia, Estrogen |
| | resistance, Mercury Poisoning |
| Asthma | Coughing, wheezing, shortness of breath, cough with phlegm, chest tightness, |
| | chills, acute episodes, fever, dry cough, anxiety |
| Bladder Cancer | Pain, blood in urine, abdominal pain, weakness, urinary retention, fever, lethargy, |
| | flank pain, infection, fatigue, pain during urination, discomfort, bowel |
| | obstruction, low blood pressure |

### A.1.5. Keyword Extraction

As part of the data modeling process according to at least some embodiments, an additional keyword extraction operation 114 may be performed. For purposes of subsequent model finetuning, which is described in more detail below, keywords may be extracted from an additional dataset comprising general conversation data. For example, the additional dataset may be the publicly available TaskMaster dataset, which contains conversations between a client and an agent. In the TaskMaster dataset conversations, the client requests the agent to perform specific tasks, such as making reservations, booking movie tickets, etc., and the agent's responses are related to the client's requests. Since the goal for the synthetic doctor-patient conversation generation model is to generate conversations, finetuning the synthetic doctor-patient conversation generation model using general conversation data as an intermediate step can help to improve the ultimate conversation generation performance of the synthetic doctor-patient conversation generation model.

In one embodiment, the keyword extraction operation 114 can be performed at random turns of the various simulated conversations present in the general conversation dataset using a sentence transformer model such as the publicly available sentence-transformers/all-MiniLM-L6-v2 model trained for mapping sentences and paragraphs to a dimensional vector space. The sentence transformer model can then be used in a keyword extraction library, such as but not limited to KeyBERT, to perform tasks such as keyword extraction. So that the extracted keywords are as diverse as possible with no overlap therebetween, a diversity score may be set relative to the keyword extraction process. In one example, the diversity score may be set at 1.5. Maximal marginal relevance (MMR) can also be utilized to avoid keyword redundancy. The extracted keywords can serve as topics during subsequent synthetic doctor-patient conversation training.

As may be observed in Table 5 below, because the utilized sentence transformer model is trained on general conversation data, extracted keywords can be generic rather than medical-specific keywords. For example, a first random conversation turn represented in Table 5 is related to reserving a table (e.g., for dinner) at some establishment and includes a stated preference for a table location that is other than at the bar. From this dialogue, the sentence transformer model extracts the keywords “table,” “7 pm,” and “bar.” While none of extracted keywords are medical-specific keywords, the extracted keywords can nonetheless be used in a downstream model finetuning process and can help to guide the synthetic doctor-patient conversation generation model toward generating synthetic conversation data in a desired conversational format.

TABLE 5

| Dialogue turn | Keywords |
|---|---|
| Person1: That's great. So I need a table for tonight at 7 pm for 8 | [‘table’, ‘7 pm’, ‘at the |
| people. We don't want to sit at the bar, but anywhere else is fine | bar’] |
| Person2: Yes, for me and a friend so two tickets please | [‘two tickets’] |
| Person1: I want to order a pizza from Bertuccis in Chelmsford, | [‘Bertuccis’, |
| MA | ‘Chelmsford, MA’] |

All the foregoing data modeling operations can be used to prepare the data required for the control parameters (TOPICS/Symptoms and ENTITIES) that will act as conversation drivers for the synthetic doctor-patient conversation generation process. The NER using a pre-trained model(s) data modeling operation 106, the post-processing of NER results data modeling operation 108, and the medical NER data modeling operation 110, are also directed to downstream final-fold finetuning of the synthetic doctor-patient conversation generation model over the primary doctor-patient conversation data sourced from one or more sets of doctor-patient conversation data such as the PriMock57 and Nature datasets. As described previously, the symptom determination data modeling operation 112 also serves to produce seed data for use in a subsequent inference phase of the synthetic doctor-patient conversation generation model. The keyword extraction operation 114 is further directed to an intermediate-fold (e.g., second-fold) finetuning operation that is designed to train the synthetic doctor-patient conversation generation model relative to conversation styles.

### A.2. Input Data Preparation

Referring still to FIG. 1, it may be observed that after the data modeling operations 106, 108, 110, 112, 114 described above have been completed, multiple control parameters can be used in conjunction with a specialized algorithm in an input data preparation operation 116 that further prepares the input data for use in a model training operation 118 and a model inference operation 120. An evaluation of the model output 122 resulting from model inference may also be performed.

Preferably, the trained synthetic doctor-patient conversation generation model is not made to output an entire synthetic conversation all at once. Instead, the trained synthetic doctor-patient conversation generation model may be designed to approach the synthetic doctor-patient conversation generation process in a turn-by-turn manner, where a first sentence or paragraph that is attributed to a first speaker (e.g., a doctor) is initially generated, and the sentence or paragraph is then posted and included as part of the input data to the next sentence or paragraph generation operation. The trained synthetic doctor-patient conversation generation model can then generate, using the input data that includes the previously generated first sentence or paragraph, a second sentence or paragraph that is attributed to a second speaker (e.g., a patient). The second sentence or paragraph can also be posted and included as part of the input data to the next (third) sentence or paragraph generation operation, which will again be attributed to the doctor given the turn-by-turn synthetic doctor-patient conversation generation technique employed by the synthetic doctor-patient conversation generation trained model. This back-and-forth generation of synthetic doctor and patient dialogue can continue until all the conversation turns resulting from a given input dataset have been generated.

Various control parameters may be utilized to control the flow of the synthetic doctor-patient conversation generation output of a trained synthetic doctor-patient conversation generation model. For example, control parameters may determine to which speaker generated synthetic dialogue should be attributed, what the turn length should be, etc. Various control parameters that can be used to control the flow of synthetic doctor-patient conversation generation output of a trained synthetic doctor-patient conversation generation model according to one embodiment of a knowledge graph synthetic doctor-patient conversation generation process, are listed below:
1. TOPICS
2. ENTITIES
3. SPEAKER
4. TURN_LENGTH
5. REMAINING_TURNS
6. CONTEXT

The TOPICS control parameter can be used to set the context of a generated synthetic doctor-patient conversation. In this example, the TOPICS terminology is employed instead of SYMPTOMS due to approaches used relative to model finetuning, which is described in more detail below. The ENTITIES control parameter is associated with each conversation/dialogue turn, and has been described in detail above relative to the various data modeling operations performed prior to input data preparation. The SPEAKER control parameter can be used to identify the person in a next generated sentence. The TURN_LENGTH control parameter can be used to define the length of the turns within a given conversation. The REMAINING_TURNS control parameter can be used to specify the number of turns that are remaining in a given conversation. The CONTEXT control parameter contains a previously generated/uttered statement, which is in turn, used to generate a next sentence.

Of the six listed control parameters associated with this embodiment, the two control parameters that are most responsible for guiding the flow of conversation generation are the TOPICS (SYMPTOMS) control parameter and the ENTITIES control parameter. As mentioned above, the term TOPICS is used instead of SYMPTOMS because a second fold of a multi-fold fine tuning process employed during model training does not include a medical context that can provide symptoms. The TOPICS act as symptoms at final model inference. For clarity the TOPICS control parameter may also be written below as Symptoms/TOPICS.

An algorithm for preparing the input data can be defined based on the control parameters. One embodiment of an input data preparation algorithm is provided below:
1. Select a maximum of 20 unique Symptoms/TOPICS that are detected previously (or less than 20 if the number of Symptoms are low) and concatenate the Symptoms using the pipe (|) symbol.
2. Follow the below steps to prepare an input dataset and an output dataset that can be used to train the finetuning (e.g., SciFive) model.
3. For each dialogue turn appearing in the data:
   a. Add a constant prepared Symptoms/TOPICS as an initial text.
   b. Concatenate the ENTITIES sampled for the current turn using the symbol (<∥>) and Add this to the Symptoms/TOPICS.
   c. Based on the turn, add the SPEAKER and TURN_LENGTH to the text.
   d. Determine REMAINING_TURNS based on the number of conversations that happen between a doctor and patient, which is added as part of this input.
   e. Append the previous dialogue as CONTEXT, except for the 1st turn where there is no previous dialogue, in which case pass an empty string for CONTEXT.
   f. Set Target sequence=Next Dialogue, or end of dialogue [EOD] if the current turn is the last dialogue.
4. At each subsequent dialogue, reduce the remaining turns such that as the conversation reaches its end, the REMAINING_TURNS becomes 0.
5. At each dialogue turn:
   a. Check if the whole input length to the model is greater than a max_input_length (or maximum tokens_len) (e.g., 2048 tokens).
   b. If yes, recursively remove conversation turns from the CONTEXT one-by-one from the beginning until the max_input_length limit is satisfied.
   c. If no, perform no processing related to the max_input_length limit and add the generated turn, including any CONTEXT dialogue, directly.
   d. Append the dialogue of any new CONTEXT to the dialogue of any preceding CONTEXT and use combined dialogue as CONTEXT in next turn.

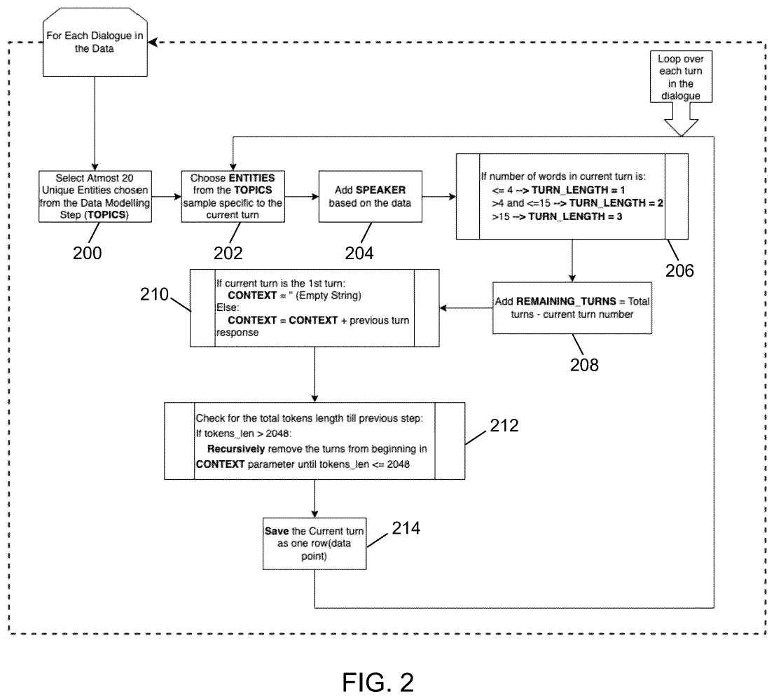

In conjunction with the above description, a flowchart representing execution of the input data preparation algorithm for each conversation turn in a given conversation/dialogue of the input data is presented in FIG. 2. As indicated at block 200 of the flowchart, in this particular example a maximum of 20 unique symptoms (TOPICS) are selected for a given conversation and concatenated. The symptoms are previously deduced, such as by performing the earlier described symptom determination data modeling operation 112. Once the symptoms have been selected, input data for finetuning the doctor-patient conversation generation model can be constructed.

As represented in FIG. 2, for each dialogue turn, execution of the input data preparation algorithm will result in a TOPICS constant appearing as the initial text of the constructed data. At block 202, ENTITIES are sampled from the TOPICS at each turn. The ENTITIES are concatenated and added after the TOPICS. The SPEAKER designator is added to the text of the constructed data at block 204. The SPEAKER for a given turn is normally the doctor or the patient having the conversation, and may alternate from turn-to-turn.

The TURN-LENGTH control parameter and its associated value can be added to the text of the constructed data as indicated at block 206. The TURN_LENGTH according to this embodiment of the input data preparation algorithm is a number from 1-3 and is determined by the number of words in a given turn. For example, as used in this embodiment of the input data preparation algorithm, the TURN_LENGTH=1 when the number of words in a turn is less than or equal to 4, the TURN_LENGTH=2 when the number of words in a turn is between 4 and 15, and the TURN_LENGTH=3 when the number of words in a turn is greater than 15. The TURN_LENGTH control parameter can be defined differently in other embodiments.

Next, a REMAINING_TURNS indication and its associated value can be added to the text of the constructed input data at block 208. The value of the REMAINING_TURNS control parameter for a given conversation turn depends on the total number of conversation turns associated with a given conversation and the number of conversation turns that have already been completed. The value of the REMAINING_TURNS control parameter may be randomly chosen and assigned to a conversation at the very beginning thereof. The value of the REMAINING_TURNS control parameter will then decrease by 1 after each turn until it reaches zero.

The CONTEXT control parameter contains a previously generated/uttered statement, which is in turn, used to generate a next sentence. The CONTEXT control parameter and its associated value can be added to the text of the constructed input data as indicated at block 210. As indicated, if the current conversation turn is the first conversation turn, the value of the CONTEXT control parameter can be an empty string, as the CONTEXT control parameter can have no value prior to any data generation by the doctor-patient conversation generation model. For subsequent turns, however, the value of the CONTEXT control parameter is the cumulative CONTEXT value from the prior turn plus the value of an immediately preceding target sequence, where the target sequence is either a next dialogue, or and end of dialogue [EOD] indicator if the current turn is the last dialogue, and is described in more detail below. For example, at a fifth turn of a given conversation, the value of the CONTEXT control parameter should be the cumulative dialogue of the target sequences of the first through the fourth conversation turns.

Following is an example doctor-patient conversation generation model input resulting from an input data preparation (construction) operation 116 performed in accordance with the control parameters of the input data preparation algorithm described above:

Example Input

″[TOPICS] <symptom values> [ENTITIES] <entity values> [SPEAKER]
<doctor/patient>
[TURN_LENGTH] <length of the turn>[REMAINING_TURNS]
<number of turns left>
[CONTEXT] <previous dialogues if available>”

Further execution of the input data preparation algorithm can also result in a check of the total tokens length, as is represented at block 212 of FIG. 2. More specifically, execution of the input data preparation algorithm can cause an evaluation of the input data length from the initial occurrence of the TOPICS constant through the addition of the CONTEXT control parameter and its associated value (dialogue). A maximum allowable input length (max_input_length) may be set for this purpose. In the embodiment presented herein, the maximum input data length for the model is set to 2,048 tokens to coincide with the maximum input data length of the pre-trained models used during the data modeling phase of the synthetic doctor-patient conversation generation process. The maximum input data length for the model may be different in other embodiments. If the input data length is greater than the allowable maximum input data length, dialogue turns may be removed from the CONTEXT starting from the beginning of the conversation until the input data length is equal to or less than 2,048 tokens (in this example), and as long as the conversation turn removal process does not result in the total number of conversation turns becoming less than two.

As represented in FIG. 2, the data associated with the current conversation turn can then be saved at block 214 as one row (data point). The input data preparation algorithm can subsequently be repeatedly executed until the dialogue for all the conversation turns of the given conversation have been constructed. The resulting input data can be used to train the doctor-patient conversation generation model, such as through multi-stage finetuning, as described in more detail below.

### A.3. Model Training

Subsequent to its construction as described above, the input data can be used to train the synthetic doctor-patient conversation generation model. According to at least some embodiments, a pre-trained model having a transformer-encoder-decoder architecture can serve as the base (untrained) model for the synthetic doctor-patient conversation generation model. One example of a pre-trained model with such a transformer-encoder-decoder architecture is the SciFive model. The SciFive model includes a transformer-encoder-decoder architecture with some self-attention and multi headed layers, and is also a variant of the Google® T5 model. The SciFive model is incrementally trained on the T5 model. The T5 model has been pre-trained using the PubMed Abstracts, which is a biomedical corpus. Consequently, the SciFive model is ideally suited for use as a base model for the synthetic doctor-patient conversation generation model because the SciFive model is inherently capable of understanding/interpreting biomedical vocabulary. Various other models may also serve as a base model for the synthetic doctor-patient conversation generation model in other embodiments. However, many such models, including the model that is associated with the T5 model by default, are not capable of understanding/interpreting biomedical vocabulary.

Finetuning of the transformer-encoder-decoder architecture base model to produce the synthetic doctor-patient conversation generation model may be performed in multiple stages according to various embodiments, although multistage finetuning is not required in all embodiments. The benefit of utilizing multistage finetuning is based on the hypothesis that applying an intermediate (e.g., second stage) finetuning operation to previously finetuned medical data used for the specific purpose of generating doctor-patient conversations, as well as to non-medical data used for the more basic purpose of generic dialogue generation, will result in a trained synthetic doctor-patient conversation generation model that adapts better to both the target problem (doctor-patient conversation generation) and to the business domain (generic dialogue generation). An analysis of key indicators during evaluation of the performance of a doctor-patient conversation generation model trained using such multistage finetuning proved this hypothesis to be correct, as model performance was better when multiple finetuning stages were utilized.

Various possible stages of one embodiment of a multistage model finetuning process are set forth and described below. In this embodiment, the multistage model finetuning process includes three separate finetuning stages (folds), but it may be possible to utilize a fewer or lesser number of finetuning stages in other embodiments.

### A.3.1. First-Fold Finetuning

A first stage (first-fold) finetuning process may be a simplistic finetuning process. For example, the first-fold finetuning process may be designed simply to align the synthetic doctor-patient conversation generation model to capture the intricacies of the medical terms encountered in the medical data domain, and also to train the synthetic doctor-patient conversation generation model on the types of answers that will be expected to be given when a specific type of question is posed to the model. In one non-limiting embodiment, the first-fold finetuning process is undertaken using a medical question answering dataset that can be used for this purpose. In at least some embodiments, the publicly available MedQA, PubMedQA, MMLU, and MedMCQA datasets may be used individually or in any combination for the medical question answering dataset. The combined data can then be used to finetune the synthetic doctor-patient conversation generation model. A goal of the first-fold finetuning process is for the synthetic doctor-patient conversation generation model to become more familiar with the ultimate goal of generating synthetic doctor-patient conversations.

In some embodiments, the first-fold finetuning process may simply involve asking the synthetic doctor-patient conversation generation model a question and causing the synthetic doctor-patient conversation generation model to answer the question. This process can be used to enhance the medical entity vocabulary of the synthetic doctor-patient conversation generation model, because although the model with a transformer-encoder-decoder architecture such as the SciFive model used in this embodiment as the base model for the synthetic doctor-patient conversation generation model is pre-trained on a biomedical corpus, biomedical entities are somewhat different from medical entities. For example, where a conversation in the medical domain may involve diseases and symptoms, a conversation in the biomedical domain may involve terminology related to the human body other than diseases and symptoms, such as the names of body parts, or references to bones, tissue, etc. Consequently, a goal of the first-fold finetuning process can be imparting the synthetic doctor-patient conversation generation model with medical terminology knowledge to go along with the biomedical terminology knowledge already present in and gained from the model with a transformer-encoder-decoder architecture such as the SciFive model. The first-fold finetuning process results in a first-finetuned synthetic doctor-patient conversation generation model.

### A.3.2. Second-Fold Finetuning

A second-fold finetuning process can follow the first-fold finetuning process to make the synthetic doctor-patient conversation generation model more adept at following the general context of conversations (i.e., conversation styles) and understanding the control parameters. Because a goal of the second-fold finetuning process is merely to train the synthetic doctor-patient conversation generation model on general conversation flow, the input data used in the second-fold finetuning process is not required to be a medical-specific dataset. Rather, the input data may be a publicly available open-source general context dataset, or a similar data source.

In at least some embodiments, second-fold finetuning may follow the strategy of the input data preparation process described above, and may be guided and controlled by the same input data preparation algorithm and control parameters to generate data in the same format, but from a general (not medical-specific) conversation. One example of a random, general conversation between two persons appears below as Example Conversation 1A, which is annotated to identify speakers and conversation turns.

Example Conversation 1a (without Turn Splitting)

### Dialogue

Person1: Hi I would like to see if the Movie What Men Want is playing here. \n Person2: Yes it's showing here would you like to purchase a ticket?\n Person1: Yes, for me and a friend so two tickets please, Person2: Okay. \n Person1: What time is that moving playing today?\n Person2: That movie is showing at 4, 5, and 8 μm. In Person1: Okay. Is there anymore movies showing around 8 pm\n Person2: Yes, showing at 8 pm is Green Book. \n Person1: What is that about?\n Person2: It's about two men dealing with racism. In Person1: Oh, no can you recommend anything else?\n Person2: What do you like?\n Person1: Well I like movies that are funny. In Person2: Like comedies?\n Person1: Well no I like action as well. In Person2: Okay. How to train your dragon is playing at 8 pm. In Person1: Okay can i get two tickets for that?\n Person2: So you want me to cancel the tickets for What men want?\n Person1: Yes please. In Person2: Okay, no problem. \n Person1: How much will this cost. In Person2: You said two adult tickets?\n Person1: Yes. In Person2: Okay, that will be $20.80\n Person1: Okay. In Person2: Anything else I can help you with?\n Person1: Yes can i bring my own food to theater. In Person2: No, sorry you have to purchase food in the lobby. In Person1: Okay that is fine.

Providing the dialogue appearing in Example Conversation 1A as input to the second-fold finetuning operation can result in the turn-by-turn data output appearing in Example Conversation 1B below. As may be observed, because the second-fold finetuning process is guided by the same input data preparation algorithm and control parameters, the data appearing in each turn of Example Conversation 1B has the same format as the data in the Example Input shown above relative to the input data preparation process. For purposes of brevity, only the first seven turns of the thirty turns of Example Conversation 1A are represented in Example Conversation 1B.

Example Conversation 1B (with turn splitting)

| Dialogue | Target |
|---|---|
| [TOPICS] adult tickets \| movie showing 8pm \| food theater sorry \| help yes | Hi I would like to see if |
| bring \| funny like comedies \| train dragon playing \| want cancel \| green | the Movie What Men |
| book \| okay time \| problem cost said \| dealing racism \| oh recommend | Want is playing here. |
| [ENTITIES] What Men Want [SPEAKER] Person 1 [TURN_LENGTH] 3 | |
| [REMAINING_TURNS] 29 [CONTEXT] | |
| [TOPICS] adult tickets \| movie showing 8pm \| food theater sorry \| help yes | Yes it's showing here |
| bring \| funny like comedies \| train dragon playing \| want cancel \| green | would you like to |
| book \| okay time \| problem cost said \| dealing racism \| of recommend | purchase a ticket? |
| [ENTITIES] {SPEAKER} Person 1 [TURN_LENGTH] 3 [REMAINING_TURNS] 28 | |
| [CONTEXT] Person1: Hi I would like to see if the Movie What Men Want is | |
| playing here. EOS | |
| [TOPICS] adult tickets \| movie showing 8pm \| food theater sorry \| help yes | Yes, for me and a |
| bring \| funny like comedies \| train dragon playing \| want cancel \| green | friend so two tickets |
| book \| okay time \| problem cost said \| dealing racism \| of recommend | please |
| [ENTITIES] two <\|\|> two tickets [SPEAKER] Person2 [TURN_LENGTH] 2 | |
| [REMAINING_TURNS] 27 [CONTEXT] Person1: Hi I would like to see if the | |
| Movie What Men Want is playing here. EOS Person2: Yes its showing here | |
| would you like to purchase a ticket? EOS | |
| [TOPICS] adult tickets \| movie showing 8pm \| food theater sorry \| help yes | Okay. |
| bring \| funny like comedies \| train dragon playing \| want cancel \| green | |
| book \| okay time \| problem cost said \| dealing racism \| of recommend | |
| [ENTITIES] [SPEAKER] Person2 [TURN_LENGTH] 1 [REMAINING_TURNS] 26 | |
| [CONTEXT] Person1: Hi I would like to see if the Movie What Men Want is | |
| playing here. EOS Person2: Yes it's showing here would you like to purchase | |
| a ticket? EOS Person1: Yes, for me and a friend so two tickets please EOS | |
| [TOPICS] adult tickets \| movie showing 8pm \| food theater sorry \| help yes | What time is that |
| bring \| funny like comedies \| train dragon playing \| want cancel \| green | moving playing today? |
| book \| okay time \| problem cost said \| dealing racism \| oh recommend | |
| [ENTITIES][SPEAKER] Person2 [TURN_LENGTH] 2 [REMAINING_TURNS] 25 | |
| [CONTEXT] Person1: Hi I would like to see if the Movie What Men Want is | |
| playing here. EOS Person2: Yes it's showing here would you like to purchase | |
| a ticket? EOS Person1: Yes, for me and a friend so two tickets please EOS | |
| Person2: Okay. EOS | |
| [TOPICS] adult tickets \| moving showing 8pm \| food theater sorry \| help yes | That movie is showing |
| bring \| funny like comedies \| train dragon playing \| want cancel \| green | at 4. 5, and 8pm. |
| book \| okay time \| problem cost said \| dealing racism \| oh recommend | |
| [ENTITIES] 4 <\|\|>5<\|\|>8pm [SPEAKER] Person2 [TURN_LENGTH] 2 | |
| [REMAINING_TURNS] 24 [CONTEXT] Person1: Hi I would like to see if the | |
| Movie What Men Want is playing here. EOS Person2: Okay. EOS Person1: | |
| What time is that moving playing today? EOS | |
| [TOPICS] adult tickets \| movie showing 8pm \| food theater sorry \| help yes | Okay. Is there anymore |
| bring \| funny like comedies \| train dragon playing \| want cancel \| green | movies showing |
| book \| okay time \| problem cost said \| dealing racism \| oh recommend | around 8pm |
| [ENTITIES] 8pm [SPEAKER] Person2 [TURN_LENGTH] 2 [REMAINING_TURNS] | |
| 23 [CONTEXT] Person1: Hi I would like to see if the Movie What men Want is | |
| playing here. EOS Person2: Yes it's showing here would like to purchase a | |
| ticket? EOS Person1: Yes, for me and a friend so two tickets please EOS | |
| Person2: Okay. EOS Person1: What time is that moving playing today? EOS | |
| Person2: That movie is showing at 4, 5, and 8pm EOS | |

For each conversation turn in Example Conversation 1B, the “Target” is the dialogue spoken by the speaker/person associated with the conversation turn, the TOPICS are derived from the above-described keyword extraction operation 114, and the ENTITIES (if present) are sampled from the TOPICS during the second-fold finetuning process at each conversation turn, are concatenated, and added after the TOPICS. Additionally, the SPEAKER is set to match the person speaking, the TURN_LENGTH value (e.g., from 1-3) is based on the length of the spoken sentence, the REMAINING_TURNS value is the randomly selected number of total turns for the conversation minus the number of preceding turns and, other than for the first turn, the CONTEXT is the previously uttered dialogue (Target). By appending the Target dialogue to the CONTEXT as shown, all the dialogue from preceding conversation turns can serve as input to a following conversation turn. The second-fold finetuning process results in a second-finetuned synthetic doctor-patient conversation generation model.

### A.3.3. Final-Fold Finetuning

A final-fold finetuning process can follow the second-fold finetuning process to make the synthetic doctor-patient conversation generation model even more adept at following the context of conversations (i.e., conversation styles) and understanding the control parameters, particularly with respect to medical-specific conversations. To that end, one or more medical-specific datasets, such as but not limited to the PriMock57 dataset and the Nature dataset, are preferably used in the final-fold finetuning process.

In at least some embodiments, final-fold finetuning may follow the strategy of the input data preparation process and the second-fold finetuning process described above, and may be guided and controlled by the same input data preparation algorithms and control parameters to generate data in the same format from a medical-specific conversation. One example of a medical-specific conversation between two persons appears as Example Conversation 2A below, with annotations to identify speakers and turns.

Example Conversation 2A (without turn splitting)
Dialogue

“Doctor: Hello? Hi. Um, should we start? Yeah, okay. Hello how um. Good morning sir, how
can I help you this morning?\nPatient: Hello, how are you? Oh hey, um, I've just had some
diarrhea for the last three days, um, and it's been affecting me I need to stay close to the toilet.
And, um, yeah, it's been affecting my day-to-day activities.\nDoctor: Sorry to hear that. Um,
and and when you say diarrhea, what'd you mean by diarrhea? Do you mean you're going to
the toilet more often? Or are your stools more loose?\nPatient: Yeah, so it's like loose and
watery stool, going to the toilet quiet often, uh and like some pain in my, like, lower
stomach?\nDoctor: Okay. And how many times a day are you going, let's say, in the last
couple of days?

Inputting the dialogue appearing in Example Conversation 2A into the second-fold finetuning model results in the turn-by-turn data output appearing in Example Conversation 2B below. As may be observed, because the second-fold finetuning process is guided by the same input data preparation algorithm and control parameters, the data appearing in each conversation turn of Example Conversation 2B has the same format as the data in the Example Input shown above relative to the input data preparation process. For purposes of brevity, only the first five turns of the 85 turns of Example Conversation 2A are represented in Example Conversation 1B.

Example Conversation 2B (with turn splitting)

| Dialogue turn | Target |
|---|---|
| [TOPICS] pain \| infection \| cramp \| loss of appetite \| vomit \| gastroenteritis | Hello? Hi. Um, should |
| \| sweats \| smoke \| alcohol \| asthma \| feverish \| vitamins \| muscular cramp | we start? Yeah, okay. |
| \| vomiting diarrhea \| fever \| vomiting \| diarrhea \| paracetamol \| watery \| | Hello how um. Good |
| tablets [ENTITIES] [SPEAKER] Doctor [TURN_LENGTH] 3 | morning sir, how can I |
| [REMAINING_TURNS] 84 [CONTEXT] | help you this morning? |
| [TOPICS] pain \| infection \| cramp \| loss of appetite \| vomit \| gastroenteritis | Hello, how are you Oh |
| \| sweats \| smoke \| alcohol \| asthma \| feverish \| vitamins \| muscular cramp | hey, um, I've just had |
| \| vomiting diarrhea I \| fever \| vomiting \| diarrhea \| paracetamol \| watery \| | some diarrhea for the |
| tablets [ENTITIES] diarrhea [SPEAKER] Patient [TURN_LENGTH] 3 | las three days, um, and |
| [REMAINING_TURNS] 83 [CONTEXT] Doctor: Hello? Hi, Um, should we | it's been affecting me I |
| start? Yeah, okay. Hello how um. Good morning sir, how can I help you this | need to stay close to |
| morning? EOS | the toilet. And, um, |
| | yeah, it's been affecting |
| | my day-to-day activities. |
| [TOPICS] pain \| infection \| cramp \| loss of appetite \| vomit \| gastroenteritis | Sorry to hear that. Um, |
| \| sweats \| smoke \| alcohol \| asthma \| feverish \| vitamins \| muscular cramp | and when you say |
| \| vomiting diarrhea I \| fever \| vomiting \| diarrhea \| paracetamol \| watery \| | diarrhea, what'd you |
| tablets [ENTITIES] diarrhea [SPEAKER] Doctor [TURN_LENGTH] 3 | mean by diarrhea? Do |
| [REMAINING_TURNS] 82 [CONTEXT] Doctor: Hello? Hi. Um, should we | you mean you're going |
| start? Yeah, okay. Hello how um. Good morning sir, how can I help you this | to the toilet more |
| morning? EOS Patient. Hello, how are you? Oh hey, um, I've just had some | often? Or are your |
| diarrhea for the last three days, um, and it's been affecting me I need to | stools more loose? |
| stay close to the toilet. And, um, yeah, it's been affecting my day-to-day | |
| activities. EOS | |
| [TOPICS] pain \| infection \| cramp \| loss of appetite \| vomit \| gastroenteritis | Yea, so it's like loose |
| \| sweats \| smoke \| alcohol \| asthma \| feverish \| vitamins \| muscular cramp | and water stool, going |
| \| vomiting diarrhea I \| fever \| vomiting \| diarrhea \| paracetamol \| watery \| | to the toilet often, uh |
| tablets [ENTITIES] watery <\|\|> pain [SPEAKER] Patient [TURN_LENGTH] 3 | and like some pain in |
| [REMAINING_TURNS] 81 [CONTEXT] Doctor. Hello? Hi. Um, should we start? | my, like lower stomach? |
| Yeah, okay, Helow how um. Good morning sir, how can I help you this | |
| morning? EOS Patient. Hello, how are you? Oh hey, um, I've just had some | |
| diarrhea for the last three days, um and it's been affecting me I need to stay | |
| close to the toilet. And, um, yeah, it's been affecting my day-to-day | |
| activities. EOS Doctor: sorry to hear that. Um, and and when you say | |
| diarrhea, what'd you mean by diarrhea? Do you mean you're going to the | |
| toilet more often? Or are your stools more loose? EOS | |
| [TOPICS] pain \| infection \| cramp \| loss of appetite \| vomit \| gastroenteritis | Okay. And how many |
| \| sweats \| smoke \| alcohol \| asthma \| feverish \| vitamins \| muscular cramp | times a day are you |
| \| vomiting diarrhea I \| fever \| vomiting \| diarrhea \| paracetamol \| watery \| | going, lets say, in the |
| tablets [ENTITIES] [SPEAKER] Doctor [TURN_LENGTH] 2 | last couple of days? |
| [REMAINING_TURNS] 80 [CONTEXT] Doctor: Hello? Hi Um, should we start? | |
| Yeah, okay. Hello how um. Good morning sir, how can I help you this | |
| morning? EOS Patient: Hello, how are you? Oh hey, um, I've just had some | |
| diarrhea for the last three days, um and it's been affecting me I need to stay | |
| close to the toilet. And, um, yeah, it's been affecting my day-to-day | |
| activities. EOS Doctor: sorry to hear that. Um, and and when you say | |
| diarrhea, what'd you mean by diarrhea? Do you mean you're going to the | |
| toilet more often? Or are you stools more loose? EOS Patient: Yeah, so it's | |
| like loose and water stool, going to the toilet quiet often, uh and like some | |
| pain in my, like, lower stomach? EOS | |

For each conversation turn in Example Conversation 2B, the “Target” is again the dialogue spoken by the speaker/person associated with the conversation turn, the TOPICS are derived from the above-described keyword extraction operation 114, and the ENTITIES (if present) are sampled from the TOPICS during the final-fold finetuning process at each turn, are concatenated, and added after the TOPICS. The SPEAKER is again set to match the person speaking, the TURN_LENGTH value (e.g., from 1-3) is based on the length of the spoken sentence, the REMAINING_TURNS value is the randomly selected number of total conversation turns for the conversation minus the number of preceding conversation turns and, other than for the first turn, the CONTEXT is the previously uttered dialogue (Target). By appending the Target dialogue to the CONTEXT as shown, all the dialogue from preceding conversation turns can serve as input to a following conversation turn. The final-fold finetuning process results in a fully trained synthetic doctor-patient conversation generation model that is ready for inference.

### A.4. Knowledge Graph Guided Model Inference

Once the fully trained synthetic doctor-patient conversation generation model has been constructed, inference may be performed to generate a synthetic doctor-patient conversation from which the performance of the synthetic doctor-patient conversation generation model can be evaluated. Model inference in this case requires conversation starter dialogue (data). And given that the desired output of the synthetic doctor-patient conversation generation model is a synthetic doctor-patient conversation, the conversation starter data should include medical terms like those that would be used in a typical real-world doctor-patient conversation (e.g., diseases, symptoms, drugs, PHI, PII, etc.). In the previously described finetuning process, the control parameters of CONTEXT, SPEAKER and REMAINING_TURNS are based substantially on random factors and no knowledge is required to define those parameters. However, this is not true for the Symptoms/TOPICS and ENTITIES control parameters because in a medical domain there is generally a link between a symptom(s) and an associated entity (disease, drug, treatment, etc.). Furthermore, when a patient visits a doctor regarding some issue for which the patient is seeking advice/assistance, the doctor-patient conversation typically flows in a manner involving some or all the relationships between the symptoms, the diseases, and their associated entities.

Because of the relationships between symptoms, diseases, and their associated entities, medical terms, or at least symptoms/topics and entities medical terms, should not simply be selected at random and supplied to the synthetic doctor-patient conversation generation model, as such terms may not be coherent (i.e., actually related in nature). Instead, the symptoms/topics supplied to the synthetic doctor-patient conversation generation model as conversation started data during the inference process are ideally ground truth data. For example, abdominal pain may be one symptom of multiple medically-accepted symptoms of a thyroid gland disease, and all of the medically-accepted symptoms are ground truths. Consequently, when selecting symptoms/topics for inclusion in conversation starter data for a synthetic doctor-patient conversation generation model inference process, it is preferable to select symptoms/topics (e.g., diseases and symptoms, diseases and treatments) that are interrelated in the real world.

To this end, inference of a trained synthetic doctor-patient conversation generation model may be knowledge graph guided. As previously mentioned, a knowledge graph is a data structure that represents knowledge as a network of interconnected/interlinked entities and their relationships. Knowledge graphs provide a way of organizing information that is easy to query and analyze. Knowledge graphs are often used in artificial intelligence applications to improve natural language processing, machine learning, and data analytics. By representing knowledge in a structured and standardized way, knowledge graphs enable more efficient and accurate data analysis and decision-making.

Within a medical domain, a knowledge graph is a structured representation of medical knowledge, including information about medical entity relationships. Such a medical knowledge graph can be used to support a wide range of applications, such as clinical decision support, drug discovery and personalized medicine. For example, a medical knowledge graph can be used to analyze patient data and suggest personalized treatment plans based on the patient's medical history, symptoms, and genetic profile. A medical knowledge graph may also be used to identify potential drug targets and predict drug interactions, which can aid in drug discovery and development. Some examples of medical entity relationships include, without limitation: Disease-Symptom; Disease-Treatment; Disease-Cause; Disease-Prevention; Disease-Complication; Medication-Indication; Medication-Side effect; Procedure-Indication; Anatomy-Related; Medical Professional-Related; and Medical Research-Related. These examples represent only a few of the medical entity relationships that are possible. Actual medical entity relationships can vary, and the complexity of the relationships can also vary depending on the specific knowledge graph and the data sources used.

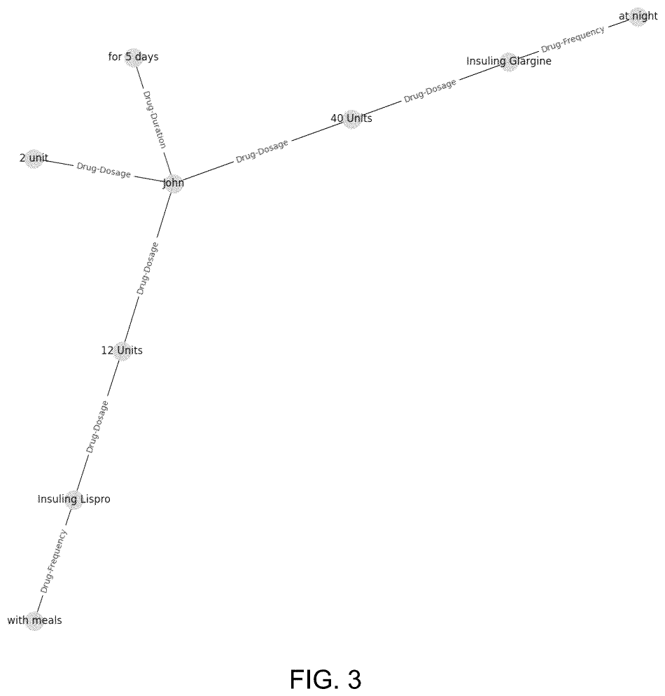

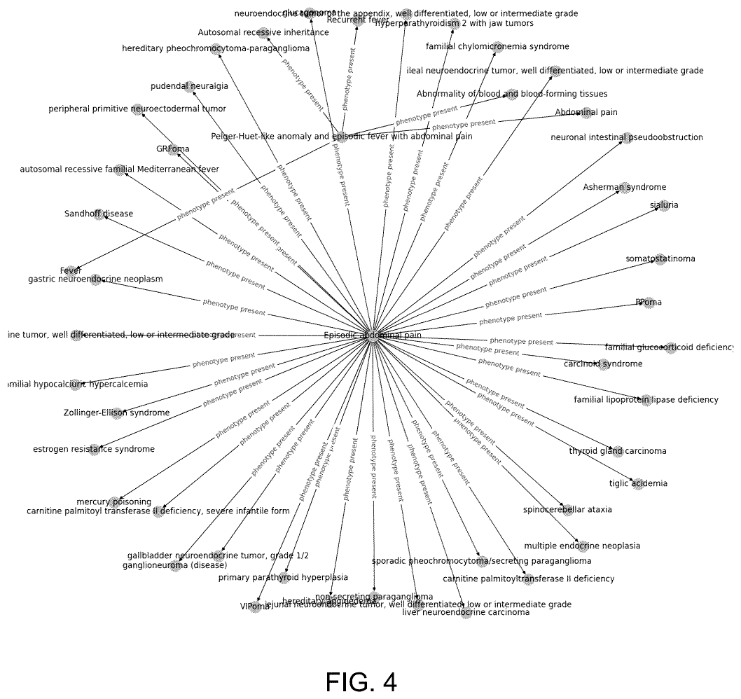

According to one embodiment of an inference process for the trained synthetic doctor-patient conversation generation model, knowledge graphs can also be combined to leverage information such as PHI, PII, treatment information, dosage or other drug information, and/or Disease-Symptom, Disease-Treatment, or other relationship information identified therein. Two examples of medical knowledge graphs are respectively shown in FIGS. 3-4. The knowledge graph example presented in FIG. 3 interconnects a plurality of entities associated with the taking of a drug by a patient (“John”), and was constructed using the previously described SciSpacy model. As indicated, the entities may be drug forms, drug dosages, drug durations, etc. The knowledge graph of FIG. 4 is a combined knowledge graph, wherein the knowledge graph of FIG. 3 has been overlaid onto a separate precision medicine multi-modal knowledge graph that was constructed using PrimeKG scripts and indicates a multitude of possible entities (diseases) that can be associated with a particular symptom. In this example, the symptom is shown to be episodic abdominal pain. Other knowledge graph embodiments may identify an array of symptoms associated with one or a plurality of different diseases.

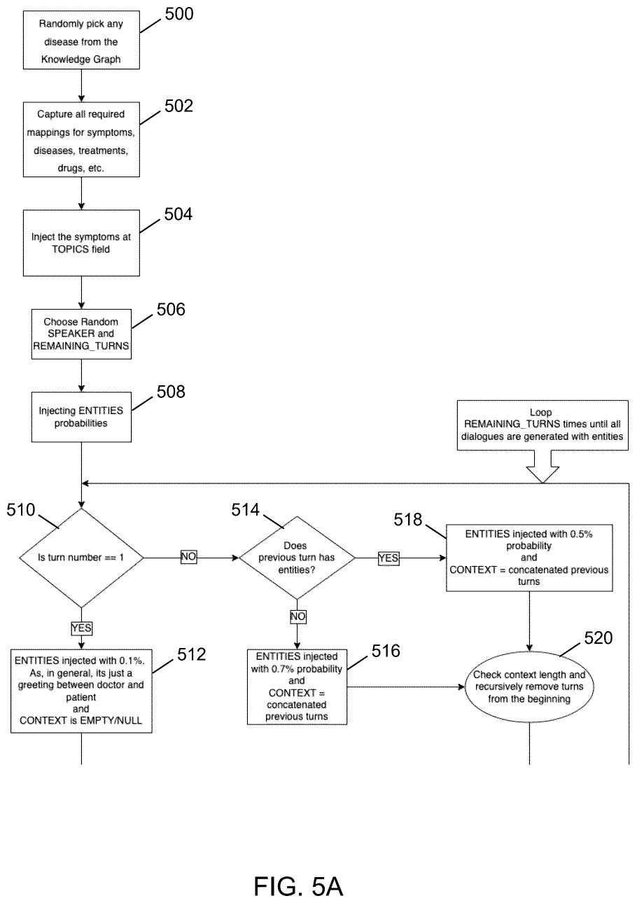

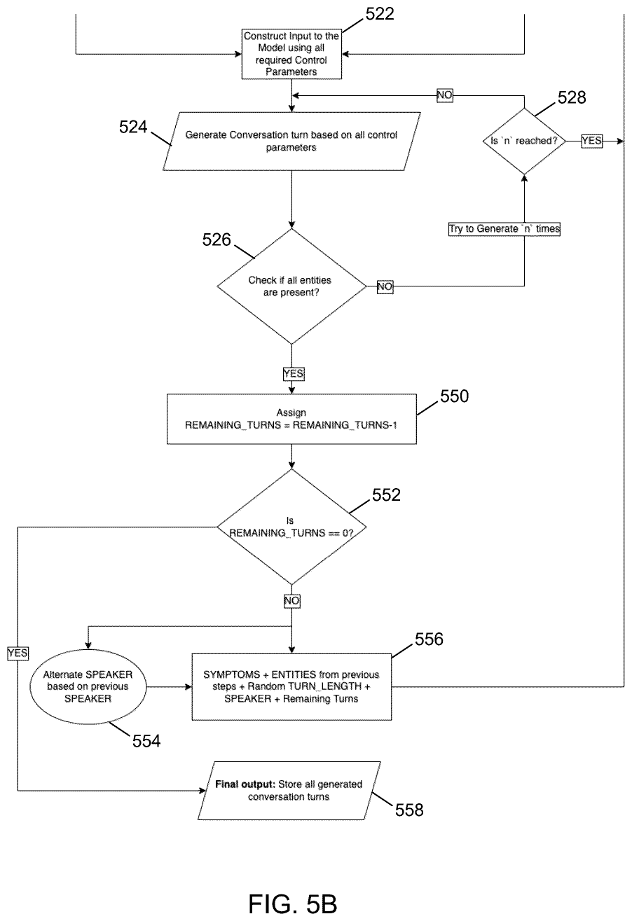

One embodiment of a synthetic doctor-patient conversation generation control algorithm is set forth below. A flowchart representing execution of the synthetic doctor-patient conversation generation control algorithm for generating the dialogue of each conversation turn of a multiple-turn synthetic doctor-patient conversation is shown in FIGS. 5A-5B.

| | |
|---|---|
| 1. | Randomly pick any disease from a constructed knowledge graph. |
| 2. | Prepare a mapping of diseases and their associated symptoms along with other properties |
| | identified in the knowledge graph. |
| 3. | Construct the TOPICS field based on the prepared mapping. |
| 4. | Choose the random SPEAKER to start the synthetic doctor-patient conversation. The |
| | speaker will alternate with each conversation turn. |
| 5. | Inject probability-based Entities into to each conversation turn, where: |

| | |
|---|---|
| a. | If turn number == 1: |

| | |
|---|---|
| i. | ENTITIES are injected with only 10% probability, as the 1st conversation |
| | turn is usually only a greeting. |
| ii. | CONTEXT in first conversation turn will be empty/null. |

| | |
|---|---|
| b. | If turn number != 1: |

| | |
|---|---|
| i. | ENTITIES are injected with 70% probability if the previous conversation |
| | turn lacks an ENTITY. |
| ii. | ENTITIES are injected with 50% probability if the previous conversation |
| | turn includes any ENTITIES. |
| iii. | CONTEXT will be the concatenation of the CONTEXT in all previous |
| | conversation turns. |

| | |
|---|---|
| 1. | If total_length > 2048: |

| | |
|---|---|
| a. | Recursively remove the previous conversation turns from |
| | CONTEXT one by one starting from the beginning. |

| | |
|---|---|
| 6. | Generate a conversation turn based on currently constructed TOPICS, ENTITIES, |
| | SPEAKER, REMAINING_TURNS, TURN_LENGTH and CONTEXT. |
| 7. | Check if all ENTITIES are present: |

| | |
|---|---|
| a. | if Yes: |

| | |
|---|---|
| i. | Continue without any changes. |

| | |
|---|---|
| b. | if No: |

| | |
|---|---|
| i. | Repeat step 6 n times, where the value of n can vary and may be user |
| | selectable. |
| ii. | If the ENTITIES are not generated even after repeating step 6 n times, try |
| | a repeat steps 5-7 using a different combination of ENTITIES. |

| | |
|---|---|
| 8. | Reduce the number of remaining conversation turns. |

| | |
|---|---|
| a. | if number of REMAINING_TURNS =v= 0: |

| | |
|---|---|
| i. | Collect all generated conversation turns. |

| | |
|---|---|
| b. | If number of REMAINING TURNS > 0: |

| | |
|---|---|
| i. | Construct a new set of control parameters based on: |

| | |
|---|---|
| 1. | Alternate SPEAKER, random TURN_LENGTH, reduced |
| | REMAINING_TURNS, and CONTEXT concatenated with the |
| | previously generated turn. |

| | |
|---|---|
| 9. | Repeat steps 5-7 until the value of the REMAINING_TURNS control parameter = 0. |

As can be understood from FIGS. 5A-5B, the above example algorithm for controlling synthetic doctor-patient conversation generation can include use of a knowledge graph, which may or may not be a combined knowledge graph. The knowledge graph can be used to select symptoms/topics to provide as conversation starter data (input) to the synthetic doctor-patient conversation generation model during an inference operation. For example, as indicated at block 500 of FIG. 5A, a disease from a knowledge graph can be randomly selected as an initial step, and as represented at block 502, a number of interrelated/mapped symptoms, diseases, treatments, etc., can thereafter be captured and used as topics in the conversation starter input to the synthetic doctor-patient conversation generation model during the inference operation. The number of captured symptoms can vary. In one embodiment, for example, twenty symptoms that are interconnected with/mapped to a selected disease may be randomly selected for use as topics in the input to the inference operation. Because the symptoms are known to be associated with the selected disease in the real world, the symptoms serve as ground-truth inputs to the synthetic doctor-patient conversation generation model.

Once the conversation starter topics have been selected, the synthetic doctor-patient conversation generation model inference process can be undertaken. Generation of a synthetic doctor-patient conversation during the inference process may be controlled by an algorithm, such as the synthetic doctor-patient conversation generation control algorithm presented above. The control algorithm includes a number of control parameters. In the example control algorithm, the control parameters are Symptoms/TOPICS, ENTITIES, SPEAKER, REMAINING_TURNS, TURN_LENGTH and CONTEXT, but may be different in other embodiments. Providing the conversation starter data as input to the synthetic doctor-patient conversation generation model causes the synthetic doctor-patient conversation generation model to generate, in a turn-by-turn manner, a synthetic doctor-patient conversation.

More specifically, the randomly selected symptoms selected from the knowledge graph(s) based on relationship/mapping to a previously randomly selected disease, can be injected (inserted) as Symptoms/TOPICS as indicated at block 504 of FIG. 5A to initiate the synthetic doctor-patient conversation generation process. The SPEAKER value and the REMAINING_TURNS value may then be randomly selected at block 506. The SPEAKER value for a given conversation turn is normally the doctor or the patient participating in the conversation, and may be randomly selected to start conversation generation. The SPEAKER value will typically alternate between doctor and patient from turn-to-turn. Likewise, the REMAINING_TURNS value can be randomly selected based on a desired synthetic doctor-patient conversation length. The REMAINING_TURNS value will decrease by one after each conversation turn. Therefore, for a conversation turn subsequent to the first conversation turn, the REMAINING_TURNS value will be the initially selected number of REMAINING_TURNS minus the number of conversation turns that have already been constructed. A synthetic doctor-patient conversation generation operation can be considered completed when the value of the REMAINING_TURNS reaches zero.

At block 508, ENTITIES are sampled from the Symptoms/TOPICS and can then be injected based on several predetermined probabilities. In this regard, it can first be determined at block 510 if the conversation turn number is equal to 1. If the conversation turn number is equal to 1, it may be assumed that the ENTITIES would rarely appear in a real-world doctor-patient conversation because, at the first turn, the conversation is just getting started and will typically comprise only a greeting, or other introductory dialogue or pleasantries. Consequently, in this example of a synthetic doctor-patient conversation generation process, it may be observed at block 512 that the ENTITIES are injected based on a probability of occurrence of 10% when the turn number is equal to 1.

Upon conclusion at block 510 that the conversation turn number is not equal to 1, it is determined at block 514 whether the previous conversation turn includes ENTITIES. As indicated at block 516, if the previous conversation turn does not include ENTITIES, then ENTITIES are injected in this example based on a probability of occurrence of 70%. As indicated at block 518, if the previous conversation turn does include ENTITIES, then ENTITIES are injected in this example based on a probability of occurrence of 50%. ENTITIES may be injected based on other probabilities of occurrence according to other embodiments of the synthetic doctor-patient conversation generation control algorithm. In any case, when ENTITIES are injected according to the present example, the ENTITIES are injected after the Symptoms/TOPICS.

It can be further observed from block 512 of FIG. 5A, that when the conversation turn is equal to 1, the CONTEXT value is empty/null because the CONTEXT is the dialogue of the previous conversation turn and there are no conversation turns prior to the first conversation turn. Conversely, as indicated in block 516 and block 518, for a given conversation turn occurring subsequent to the first conversation turn, the CONTEXT value may be the concatenated dialogue of all the preceding conversation turns. For example, at a tenth turn of a given generated synthetic doctor-patient conversation, the CONTEXT value should be the concatenated cumulative dialogue of the preceding nine conversation turns.

As indicated at block 520, once the Symptoms/TOPICS, SPEAKER, REMAINING_TURNS, and CONTEXT parameters have been injected for a given conversation turn, the total token length of the CONTEXT value (i.e., the dialogue that constitutes the CONTEXT) can be checked to ensure it does not exceed a predetermined maximum length. In this example, the maximum CONTEXT length is 2,048 tokens in view of the previous data modeling operation that set the maximum CONTEXT length of the training data at 2,048 tokens. Other maximum CONTEXT lengths may be utilized in other embodiments. As also indicated at block 520, if the CONTEXT length becomes greater than the allowable maximum CONTEXT length during the conversation generation process, conversation turns may be removed from the already generated synthetic doctor-patient conversation starting from the beginning of the conversation until the CONTEXT length is equal to or less than 2,048 tokens.

As indicated at block 522 of FIG. 5B, the Symptoms/TOPICS, ENTITIES, SPEAKER, REMAINING_TURNS, and CONTEXT parameter values form an input to the synthetic doctor-patient conversation generation model. The input is thereafter provided to the synthetic doctor-patient conversation generation model and a synthetic doctor-patient conversation turn is generated as represented at block 524.

The generated synthetic doctor-patient conversation turn can then be checked to determine if all expected ENTITIES are present, as indicated at block 526. If all the expected ENTITIES are not present, the conversation turn can be generated again and the check for all expected ENTITIES can be repeated. This process of generating a new conversation turn and checking the generated conversation turn for all expected ENTITIES can be repeated until either the expected ENTITIES are determined to be present in the generated conversation turn or a predetermined limit on the number (n) of attempts is reached. If the expected ENTITIES are still not present in the generated conversation turn after the predetermined limit on the number (n) of conversation turn generation and ENTITIES check loops has been reached, the synthetic doctor-patient conversation generation process can return upstream and repeat the ENTITIES injection operation while maintaining the current conversation turn value.

If it is determined after a synthetic doctor-patient conversation turn is generated at block 524, that all expected ENTITIES are present, the REMAINING_TURNS parameter value can be decremented by one, as indicated at block 530. The REMAINING_TURNS parameter value can then be checked as represented at block 532 to determine if the REMAINING_TURNS parameter value has reached zero. If it is determined by block 532 that the REMAINING_TURNS parameter value has not reached zero, a new synthetic doctor-patient conversation turn can be generated. When generating the new synthetic doctor-patient conversation turn, the SPEAKER parameter value may alternate from the SPEAKER identified in the preceding conversation turn to the next speaker (e.g., from doctor to patient), as is indicated at block 534. Additionally, the Symptoms/TOPICS, ENTITIES, REMAINING_TURNS, and CONTEXT parameter values, as well as the new SPEAKER parameter value, can be looped upstream where the ENTITIES can be injected relative to generation of a next conversation turn according to the probability of occurrence set forth in block 516 or block 518. This looping of control parameter values and generation of additional synthetic doctor-patient conversation turns may continue until the REMAINING_TURNS parameter value has reached zero, as determined at block 532. When the REMAINING_TURNS parameter value reaches zero, the synthetic doctor-patient conversation generation operation may be considered to be complete and the cumulative generated synthetic doctor-patient conversation turns can be considered the final output of the synthetic doctor-patient conversation generation model. All generated synthetic doctor-patient conversation turns may be stored for subsequent evaluation, as represented at block 538.

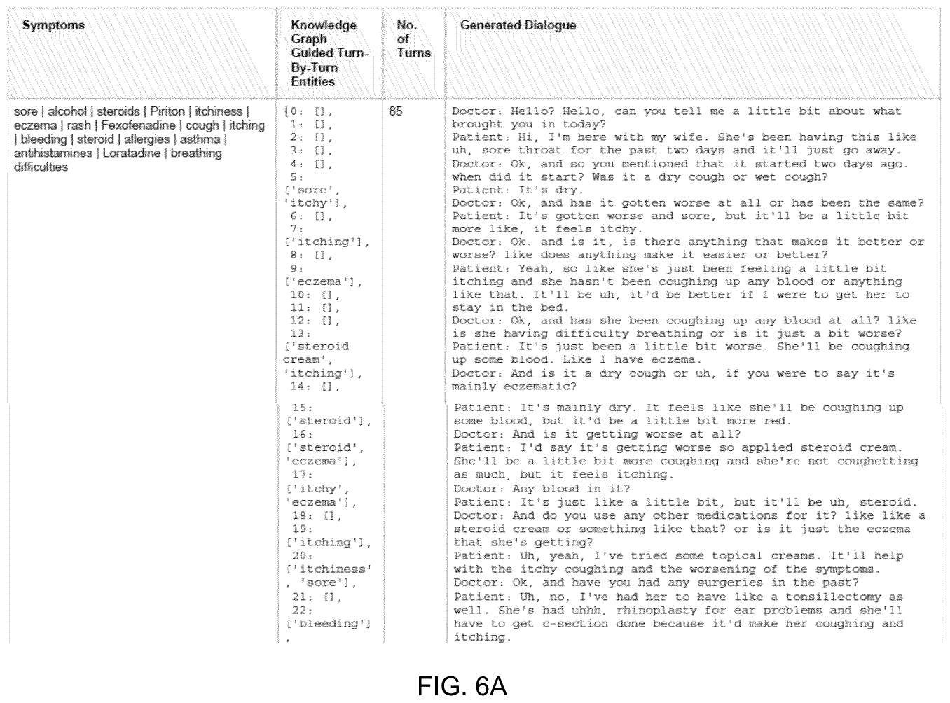

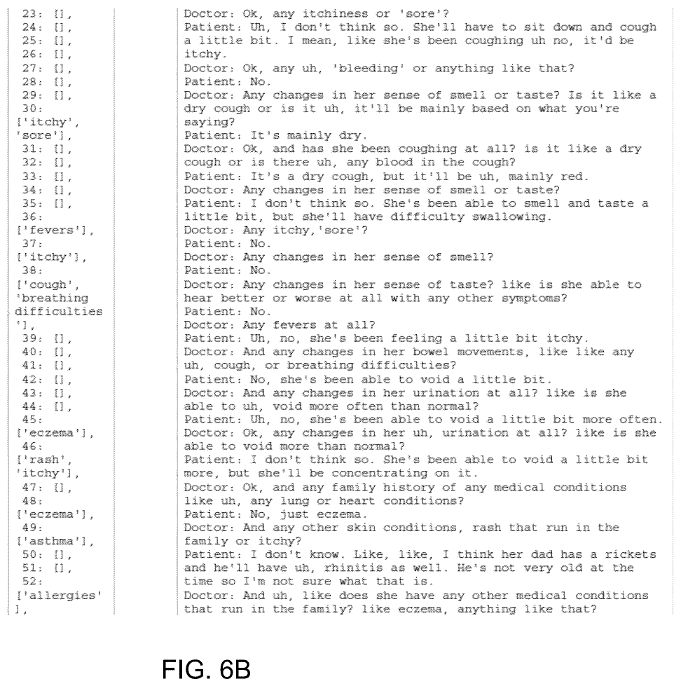

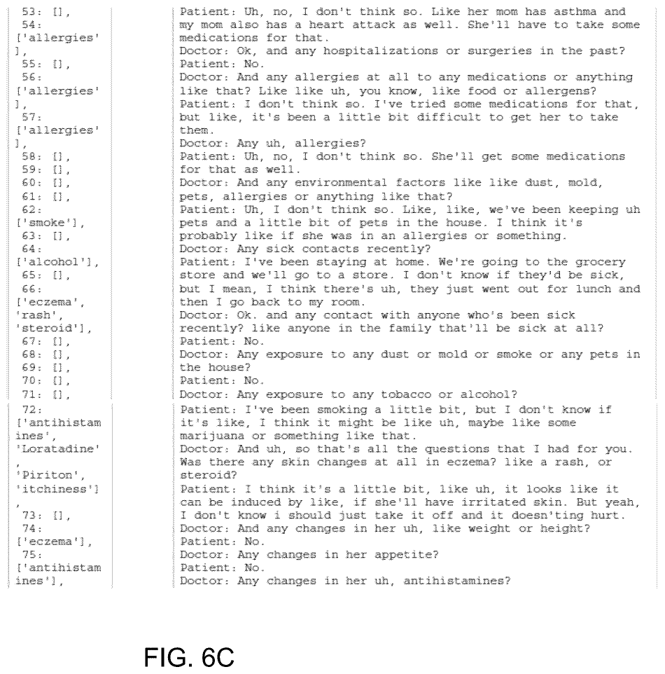

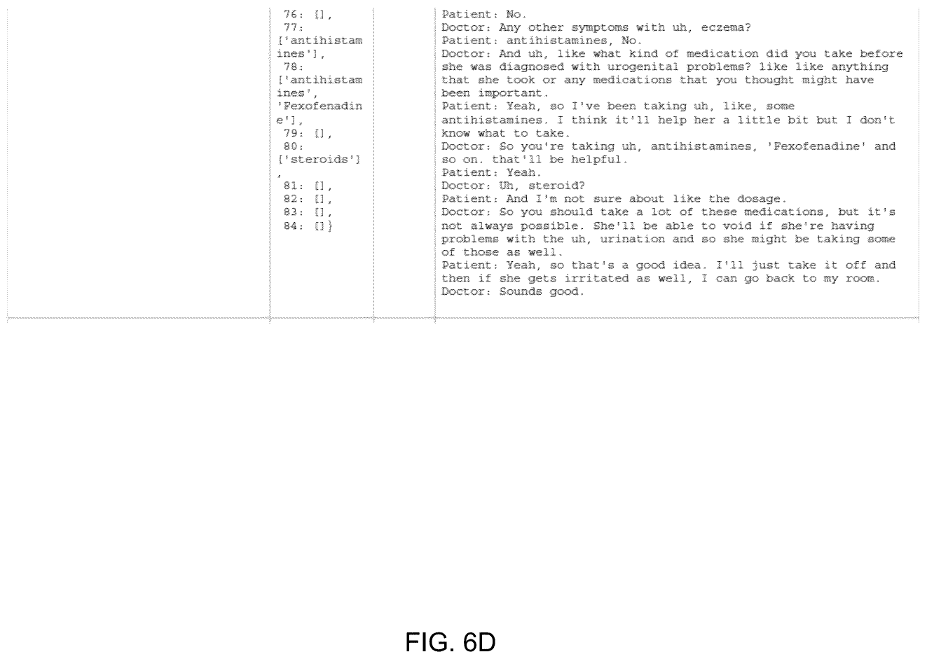

One example of a portion of a doctor-patient synthetic conversation generated by one embodiment of a synthetic doctor-patient conversation generation model under control of the synthetic doctor-patient conversation generation control algorithm described herein, is presented in table form in FIGS. 6A-6D. As represented in a first column of FIGS. 6A-6D, a plurality of symptoms are derived from a knowledge graph(s) and supplied to the synthetic doctor-patient conversation generation model as conversation starter input (seed data) at model inference. The ENTITIES sampled from the Symptoms/TOPICS at each conversation turn are listed in a second, conversation turn column. In the conversation turn column, the “0” conversation turn indicator designates the first conversation turn. The lack of any ENTITIES being associated with the first conversation turn indicates that the synthetic doctor-patient conversation generation model considered the 10% probability of occurrence ENTITIES injection control parameter to be not satisfied in this example. A third column of FIGS. 6A-6D indicates that the doctor-patient synthetic conversation generated in response to the model input has been randomly selected to have 85 total conversation turns in this example.

The final column of the table of FIGS. 6A-6D shows the doctor-patient synthetic conversation generated by the synthetic doctor-patient conversation generation model in response to the model input. The dialogue of the doctor-patient synthetic conversation appears on a turn-by-turn basis, with the doctor being selected by the synthetic doctor-patient conversation generation model as the first speaker and the SPEAKER parameter value thereafter alternating between the doctor and the patient at each conversation turn. The ENTITIES injected by the synthetic doctor-patient conversation generation model at a given conversation turn may appear in the dialogue of the following conversation turn. For example, where the synthetic doctor-patient conversation generation model injected “sore” and “itchy” ENTITIES at the fifth conversation turn, those ENTITIES appear in the sixth turn of generated synthetic conversation within the patient uttered dialogue “It's gotten worse and sore, but it'll be a little bit more like, it feels itchy.” The appearance of sampled ENTITIES in the dialogue of a generated doctor-patient synthetic conversation is based on the performance of the model, and not all sampled ENTITIES will necessarily appear in the dialogue of a generated doctor-patient synthetic conversation.

When generating the dialogue of the doctor-patient synthetic conversation, synthetic doctor-patient conversation generation model attempts to optimize on the ENTITIES and relations at each turn. This helps to control the flow of synthetic doctor-patient conversation generation, and also facilitates the addition of chosen medical CONTEXT along with ground-truth symptoms and diseases associated therewith to ensure the generated doctor-patient synthetic conversation is guided by facts.

### A.5. Evaluation of Model Performance

Once a doctor-patient synthetic conversation has been generated by the synthetic doctor-patient conversation generation model, the conversation generation performance of the model can be evaluated. Synthetic doctor-patient conversation generation model performance can be evaluated in various ways in different embodiments.

In at least some embodiments, synthetic doctor-patient conversation generation model performance may be evaluated on the basis of three metrics. One such metric may be recall-oriented understudy for gisting evaluation (Rouge), and more particularly, Rouge-C. The ROUGE-C evaluation metric operates by calculating, for each turn in a generated doctor-patient synthetic conversation, a ratio of the number of ENTITIES tokens appearing in the generated conversation dialogue to the total number of ENTITIES tokens that were sampled from the Symptoms/TOPICS. An average ratio over the entire generated doctor-patient synthetic conversation can then be determined from the calculated ratios for each of the conversation turns. The higher the average ratio, the better the synthetic doctor-patient conversation generation model performance.

A second metric of the aforementioned three metrics that can be used to evaluate synthetic doctor-patient conversation generation model performance according to this embodiment is an n-gram diversity score. The n-gram diversity score is calculated as the ratio of the number of unique n-grams (word sequences) to the total number of n-grams that are present within the entirety of a generated doctor-patient synthetic conversation. The n-gram diversity score may be calculated after stop words, punctuation, etc., has been removed from the dialogue of a generated doctor-patient synthetic conversation. N-gram diversity is related to the ability of the synthetic doctor-patient conversation generation model to generate doctor-patient synthetic conversation with proper meaning and context. Consequently, the higher the average ratio of unique n-grams to total n-grams in a generated doctor-patient synthetic conversation, the better the synthetic doctor-patient conversation generation model performed.

A third metric of the aforementioned three metrics that can be used to evaluate synthetic doctor-patient conversation generation model performance according to this embodiment is unique n-gram count. The unique n-gram count is simply a count of the total number of unique n-grams (word sequences) that appear in a generated doctor-patient synthetic conversation. Because the presence of a large number of unique n-grams tends to result in a generated doctor-patient synthetic conversation with more accurate meaning and context, the higher the count of unique n-grams in a generated doctor-patient synthetic conversation, the better the synthetic doctor-patient conversation generation model performed.

### B. Conversation Summary Guided Synthetic Doctor-Patient Conversation Generation

The synthetic doctor-patient conversation generation process embodiments described above are based on knowledge graph guidance and entity control. However, it is possible in other embodiments to instead generate synthetic doctor-patient conversations using existing doctor-patient conversation summaries as input to a synthetic doctor-patient conversation generation model. A doctor-patient conversation summary may be, for example and without limitation, a clinician's subjective, objective, assessment and plan (SOAP) notes, a patient discharge summary, or another summary of a conversation associated with a doctor-patient interaction.

A majority of the synthetic doctor-patient conversation generation model and associated synthetic doctor-patient conversation generation process embodiments described above with respect to knowledge graph guided and entity controlled synthetic doctor-patient conversation generation can also be employed when performing a conversation summary guided synthetic doctor-patient conversation generation operation. Generally speaking, the primary difference between the two techniques is that while ENTITIES sampled from Symptoms/TOPICS are used to generate a synthetic doctor-patient conversation according to the knowledge graph guided technique, existing conversation summaries are instead used to generate a synthetic doctor-patient conversation according to the conversation summary guided technique. Both techniques employ a control algorithm having various control parameters for guiding and controlling synthetic doctor-patient conversation, and a majority of the control parameters may be utilized across both techniques.

In the case of a conversation summary guided synthetic doctor-patient conversation generation process, the input data preparation procedure associated with the knowledge graph guided end entity controlled synthetic doctor-patient conversation generation process can be omitted because the input data is already present in the form of the existing doctor-patient conversation summaries. However, model training is still required.

### B.1. Model Training

According to at least some embodiments, an existing model having a transformer-encoder-decoder architecture can serve as the base (untrained) model for the synthetic doctor-patient conversation generation model used in a conversation summary guided synthetic doctor-patient conversation generation process. The SciFive model may again be used as the base model for the synthetic doctor-patient conversation generation model. As previously explained, the SciFive model is incrementally trained on the T5 model, which renders the SciFive model inherently capable of understanding/interpreting biomedical vocabulary. Various other models may also serve as a base model for the synthetic doctor-patient conversation generation model in other embodiments of a conversation summary guided synthetic doctor-patient conversation generation process. However, as already explained many such models are not capable of understanding/interpreting biomedical vocabulary.

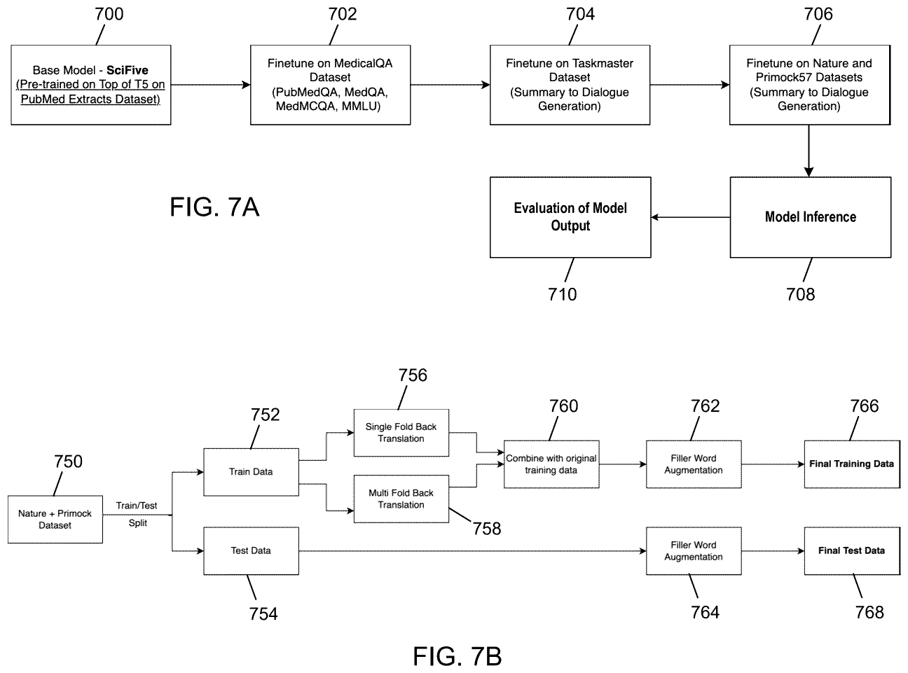

Finetuning of the transformer-encoder-decoder architecture base model to produce the synthetic doctor-patient conversation generation model may be performed in multiple stages according to various embodiments, although multistage finetuning is not required in all embodiments. One embodiment of a synthetic doctor-patient conversation generation model finetuning process is represented in the flowchart of FIG. 7A. In a similar fashion to the knowledge graph guided end entity controlled synthetic doctor-patient conversation generation process, finetuning of the doctor-patient conversation summary data may be performed in multiple stages, although multistage finetuning is not required in all embodiments.

As indicated in FIG. 7A, in one example embodiment, initial finetuning of the SciFive base model 700 for the synthetic doctor-patient conversation generation model may again be performed in three stages, but it may be possible to utilize a fewer or lesser number of finetuning stages in other embodiments. Finetuning by each of the three stages is described in more detail below.

### B.2. First-Stage Finetuning

As represented in block 702 of FIG. 7A, the first stage of finetuning the SciFive (or other encoder-decoder based model) may utilize a medical question answering dataset. Question answering in a medical domain is similar in nature to the question and answer manner in which real-world doctor-patient conversations are typically conducted. Therefore, a question answering dataset can be used to help the trained synthetic doctor-patient conversation generation model align to the task of generating answers to specific questions in the medical domain. The medical question answering dataset may again be one or more of the MedQA, PubMedQA, MMLU, and MedMCQA datasets, and the data of each of the datasets can once again be combined together and used to finetune the synthetic doctor-patient conversation generation model. In at least some embodiments, the finetuning problem may be modeled as a Seq2Seq generation problem, in accordance with the SciFive model architecture. In such a case, the questions in the question answering datasets can be used as input sequences, while the resulting answers can be used as output sequences.

### B.3. Second-Stage Finetuning

As indicated in block 704 of FIG. 7A, a second-stage finetuning process can follow the first-stage finetuning process for the purpose of improving the conversation generation performance of the synthetic doctor-patient conversation generation model. For second-stage finetuning, the first-stage finetuned synthetic doctor-patient conversation generation model can be further finetuned using an additional dataset. In at least some embodiments, the additional dataset is preferably a dataset of general conversation data. For example, the additional dataset may again be the publicly available TaskMaster dataset, which has been described above as containing non-medical conversations between a client and an agent. As is the case with knowledge graph guided synthetic doctor-patient conversation generation, finetuning the conversation summary guided synthetic doctor-patient conversation generation model on general conversation data as an intermediate step can help improve the ultimate conversation generation performance of the model. A pre-trained dialogue summarization model may be used to generate conversation summaries from the additional general conversation dataset. For example, in one embodiment a dialogue summarization model called distilbart-samsum may be used to generate conversation summaries, such as but not limited to, summaries of the client-agent conversations in the TaskMaster dataset.

Second-stage finetuning relative to conversation summary guided synthetic doctor-patient conversation generation may follow a turn-by-turn control parameter based approach to training data creation. The use of control parameters may allow variations of a conversation to be generated from the same conversation summary input. For inference, the values of the control parameters can be randomly generated. Certain control parameters in conjunction with the context of a summarized conversation can also help to drive the conversation flow and introduce chit-chat type utterances into the conversation.

The turn-by-turn control parameter based approach may be similar to the previously described turn-by-turn control parameter based approach associated with the knowledge graph guided and entity controlled synthetic doctor-patient conversation generation. For example, the control parameters used during second-stage finetuning of the conversation summary guided synthetic doctor-patient conversation generation model may include the same SPEAKER, TURN_LENGTH, REMAINING_TURNS, and CONTEXT control parameters described above with respect to knowledge graph guided and entity controlled synthetic doctor-patient conversation generation. However, a SUMMARY control parameter is substituted for the Symptoms/TOPICS and ENTITIES control parameters that are utilized relative to the knowledge graph guided and entity controlled synthetic doctor-patient conversation generation process. Further, in the case of a conversation summary guided synthetic doctor-patient conversation generation process, an instruction can be appended at each conversation summary turn to direct the synthetic doctor-patient conversation generation model how to summarize a given conversation of the additional (e.g., TaskMaster) dataset. For example, an instruction may direct the synthetic doctor-patient conversation generation model to “generate a response from the doctor given the summary and the conversation context.”

### B.4. Final-Stage Finetuning

As represented at block 706, a final-stage finetuning process can follow the second-stage finetuning process, and may correspond to the actual task of generating synthetic doctor-patient conversations. More specifically, the final-stage finetuning process can be used to better train the synthetic doctor-patient conversation generation model how to generate synthetic doctor-patient conversations from the conversation summaries. Detailed steps of one embodiment of a final-stage finetuning process are represented in the flowchart of FIG. 7B. As indicated at block 750, final-stage finetuning according to embodiments of a conversation summary guided synthetic doctor-patient conversation generation process may utilize publicly available curated doctor-patient conversation data, such as one or both of the PriMock57 dataset and the Nature dataset described above. In at least some embodiments the PriMock57 dataset may be combined with the Nature dataset to provide a dataset having approximately 330 data points comprising simulated doctor-patient conversations. The combined dataset may be split into a training dataset as indicated at block 752 and a test dataset as indicated at block 754.

Since the curated doctor-patient conversation dataset described here is very small relative to proper model training standards, at only approximately 330 data points, training of the synthetic doctor-patient conversation generation model using the curated doctor-patient conversation dataset may result in extreme overfitting. Consequently, a series of operations may be undertaken to reduce the effect of overfitting by the synthetic doctor-patient conversation generation model during finetuning with the curated doctor-patient conversation dataset.

### B.4.1. Back Translation Augmentation

A first operation that may be undertaken to reduce the effect of overfitting by the synthetic doctor-patient conversation generation model is back translation augmentation. Back translation is a process of translating text from one language to another and then translating the text back to the original language. Back translation can help paraphrase text, thus enabling enhanced language diversity. Back translation works for paraphrasing because one piece of text can be written in multiple ways when going from one language to another. Considering the small number of data points presented by the curated doctor-patient conversation dataset, and given that most of the data in the curated doctor-patient conversation dataset comes from a single source when the dataset used is a combination of the combined PriMock57 and Nature dataset, back translation can be utilized to enhance the diversity of the language in the curated doctor-patient conversation dataset. For example, back translation can be used to generate new types of dialogue utterances, such as dialogue utterances that are written in a in a different manner from an original conversation or an original dialogue of the curated doctor-patient conversation dataset. Back translation can also help to remove inconsistencies appearing in the original data of the curated doctor-patient conversation dataset, such as some common filler words.

In some embodiments, two types of back translation may be performed on the training dataset. As indicated at block 756 of FIG. 7B, one type of back translation that may be performed is single fold back translation. In single fold back translation, text written in a first language (e.g., English) is translated to another language, and then translated back to the first language. As indicated at block 758 of FIG. 7B, another type of back translation that may be performed on the training dataset is multi-fold back translation. In multi-fold back translation, text written in a first language (e.g., English) is translated through a series of other languages before being translated back to the first language. Multi-fold back translation may result in even greater diversity of language than single fold back translation. Back translation can be performed on both conversation dialogues and conversation summaries, where the conversation dialogues may be back translated one turn at a time and the summaries may be back translated one sentence at a time. Multiple instances of back translated training datasets can be produced and subsequently appended to the original training datasets, as represented at block 760.

### B.4.2 Filler Word Redistribution

As indicated at block 762 and block 764 of FIG. 7B, a second operation that may be undertaken to reduce the effect of overfitting by the synthetic doctor-patient conversation generation model is filler word redistribution. In the case of a small training dataset, such as the curated doctor-patient conversation dataset described herein, most of the filler words that occur in the conversations are likely to follow a similar distribution and to be quite similar. This can cause the synthetic doctor-patient conversation generation model to overfit to a similar pattern and thus produce conversation outputs that are limited in diversity. To improve filler word diversity according to some embodiments, both the training dataset and the test dataset can be subjected to a filler word redistribution algorithm. In addition to improving the diversity of the filler words already present in the training data, the filler word redistribution algorithm may also ensure that at least some types of filler words that are not present in the original dataset are introduced to the training dataset. Completion of the filler word redistribution process results in a final training dataset as represented at block 766 and a final test dataset as represented at block 768.

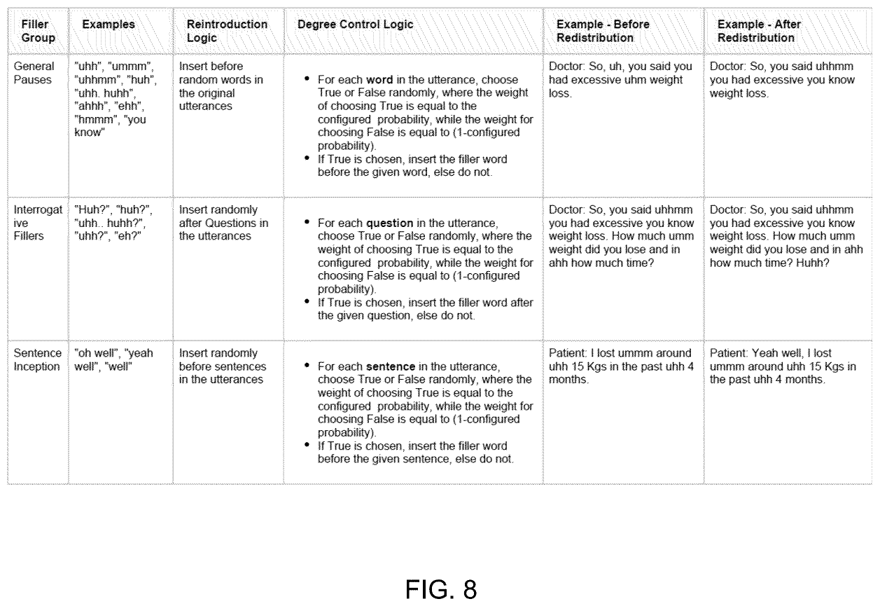

As represented in the table of FIG. 8, filler word redistribution may be accomplished in at least some embodiments by, for example, initially dividing filler words into several (e.g., three) categories (i.e., filler groups). The three filler groups may be, in at least some embodiments, general pauses, interrogative fillers, and sentence inception. As can be observed in FIG. 8, general pauses are typically common words or sounds, such as “uhh,” “ummm,” “ahhh,” “hmmm,” etc., that a speaker might make in the course of conversation, such as when a speaker is considering what to say next. Interrogative fillers are words typically injected into a conversation after a question, such as “huh?” and “eh?.” Sentence inception filler words are words typically used immediately before starting a sentence, and may include for example, words such as “well,” “yeah well,” etc.

When an input dataset has a limited variety of filler words present in the existing dialogue, the filler words can be removed and then reintroduced on the basis of the filler groups and under the guidance of the filler word redistribution algorithm. Filler word removal can be performed using, for example, a regular expression matching technique. Various possible filler word reintroduction logic examples are also indicated in the third column of the table of FIG. 8. For example, filler words may be reintroduced before random words, randomly after questions, or randomly before sentences. Various degrees of probability-based control that can be used to govern filler word reintroduction into the generated dataset(s) are similarly indicated in the fourth column of the table of FIG. 8. Several example sentences evidencing the effect of filler word redistribution are respectively shown in the fifth and sixth columns of the table of FIG. 8 for each filler word group.

### B.4.2.1. Frequent Word Replacement

When the original dataset used to generate the training dataset is small, it is possible that certain filler words (e.g., “OK”) may appear much more frequently than others and may also follow a fixed pattern. To avoid having the synthetic doctor-patient conversation generation model memorize frequently occurring filler words, and to ensure that the synthetic doctor-patient conversation generation model can understand other words that may be substituted for the frequently occurring filler words, a frequent word replacement operation can be performed.

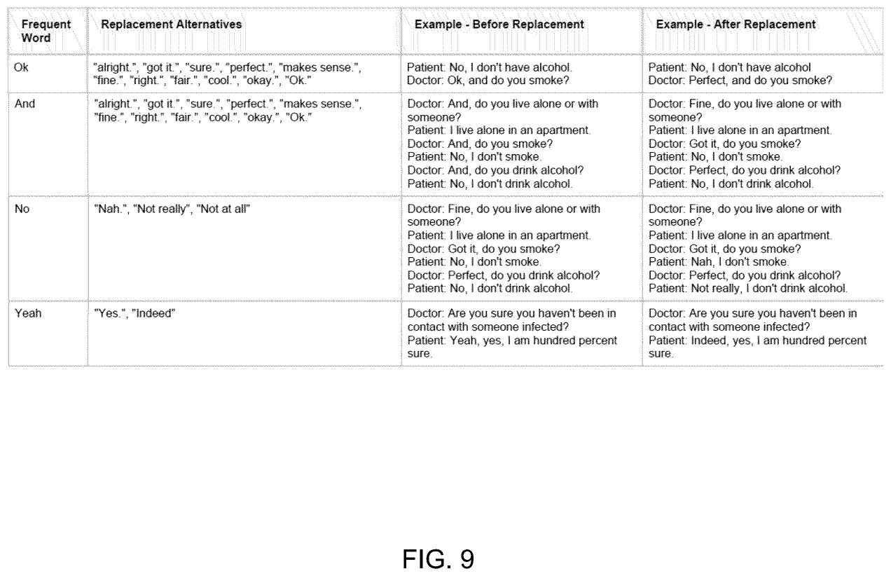

During frequent word replacement, identified frequently occurring words may be removed completely, or may be replaced with similar words. Examples of several frequently occurring words and various possible alternative replacement words are presented in the table of FIG. 9. In this example, the frequently occurring words appear in the first column of the table and are shown to be “OK,” “and,” “no,” and “yeah.” A plurality of possible alternative replacement words are shown in the second column of the table of FIG. 9 for each of the frequently occurring words. Finally, several example sentences evidencing the results of frequent word replacement are respectively shown in the third and fourth columns of the table for each frequently occurring word. The degree of frequent word replacement can be controlled using the same probability-based control logic described above and shown in the table of FIG. 9 relative to the filler word redistribution process.

### B.5. Conversation Summaries Guided Model Inference

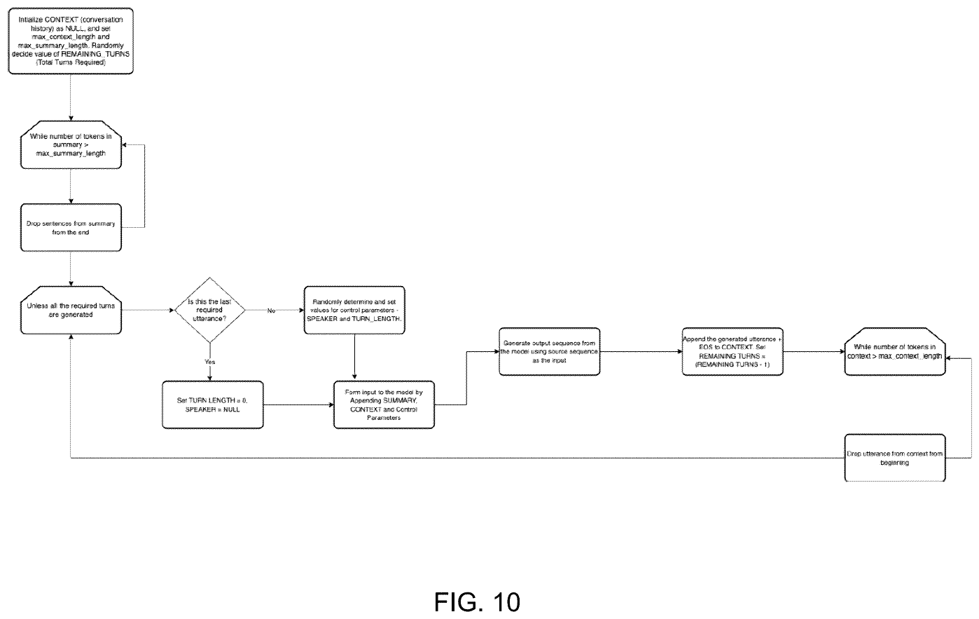

As indicated at block 708 of FIG. 7A, once the fully trained synthetic doctor-patient conversation generation model has been constructed, the trained model is ready for inference. Inference can be performed by providing the final test dataset 768 as input to the trained synthetic doctor-patient conversation generation model. Conversation summary guided model inference may be controlled by an algorithm having a number of different control parameters including, but not limited to, SPEAKER, REMAINING_TURNS, TURN_LENGTH and CONTEXT. One example of a conversation summary guided model inference process is illustrated in the flow diagram of FIG. 10. During inference, the trained conversation summary guided model will output a turn-by-turn synthetic doctor-patient conversation based on the conversation summaries in the final test dataset 768.

### B.6. Evaluation of Model Performance

Once a doctor-patient synthetic conversation has been generated by the conversation summary guided synthetic doctor-patient conversation generation model, the conversation generation performance of the model can be evaluated as indicated in block 710 of FIG. 7A. Synthetic doctor-patient conversation generation model performance can be evaluated in various ways in different embodiments of the conversation summary guided synthetic doctor-patient conversation generation process.

In at least some embodiments, synthetic doctor-patient conversation generation model performance may be evaluated on the basis of three metrics. One such metric may be Rouge-C (recall), the general operation of which is described above relative to the knowledge graph guided synthetic doctor-patient conversation generation process. In the case of a conversation summary guided synthetic doctor-patient conversation generation, the ROUGE-C evaluation metric operates by calculating, for each turn in a generated doctor-patient synthetic conversation, a ratio of the number of unique tokens (words) appearing in the generated conversation dialogue to the total number of unique tokens present in the conversation summaries of the final test dataset 768. An average ratio over the entire generated doctor-patient synthetic conversation can then be determined from the calculated ratios for each of the conversation turns. The higher the average ratio, the better the synthetic doctor-patient conversation generation model performance.

A second metric of the aforementioned three metrics that can be used to evaluate conversation summary guided synthetic doctor-patient conversation generation model performance according to this embodiment is again an n-gram diversity score. The n-gram diversity score relative to the conversation summary guided synthetic doctor-patient conversation generation process is calculated as the ratio of the number of unique n-grams (word sequences) to the total number of n-grams that are present within the entirety of a generated doctor-patient synthetic conversation. The n-gram diversity score may be calculated after stop words, punctuation, etc., has been removed from the dialogue of a generated doctor-patient synthetic conversation. The higher the average ratio of unique n-grams to total n-grams in a generated doctor-patient synthetic conversation, the better the synthetic doctor-patient conversation generation model performed.

A third metric of the aforementioned three metrics that can be used to evaluate synthetic doctor-patient conversation generation model performance according to this embodiment is referred to as clinical concept recall. Clinical concept recall is a calculation of the ratio of the number of clinical terms (disease name, medicine name, symptoms, etc.) present in a given conversation summary, to the number of such clinical terms present in a synthetic conversation generated from the conversation summary. A clinical concept recall calculation may be performed in at least some embodiments using a deep learning model, such as but not limited to, a deep learning model from Spacy. Clinical concept recall calculations may result in false positives. Such false positives can be reduced through the use of speech filtration (e.g., by extracting only nouns).

### C. Combined Knowledge Graph and Conversation Summaries Guided Synthetic Doctor-Patient Conversation Generation

As thus far described above, synthetic doctor-patient conversation generation may be either knowledge graph guided or conversation summary guided. However, it is also possible in some embodiments that a synthetic doctor-patient conversation generation model can be trained to generate synthetic doctor-patient conversations using a combined knowledge graph guided and conversation summary guided synthetic doctor-patient conversation generation process. For example, the knowledge graph guided conversation generation technique can be used to select a disease(s) from a knowledge graph and to identify a multitude or all of the mapped symptoms associated with the selected disease as entities. All the information gathered from the knowledge graph can then be combined with the information contained in the existing doctor-patient conversation summaries serving as the original dataset(s) according to the conversation summary guided synthetic doctor-patient conversation generation technique, and a synthetic doctor-patient conversation generation model may be subsequently trained on the data in the combined (enhanced) dataset. Instructions may still be included in each conversation turn of the combined training dataset. In at least one embodiment of a combined knowledge graph guided and conversation summary guided synthetic doctor-patient conversation generation process, named entities can be extracted from existing conversation summaries and a knowledge graph can then be used to find the linked entities.

Once training on the combined/enhanced dataset is complete, model inference can proceed in the manner already described above relative to either the knowledge graph guided or conversation summary guided synthetic doctor-patient conversation generation process. Likewise, an evaluation of generated conversation samples may be undertaken using any or all of the evaluation metrics described above.

In other embodiments where knowledge graph guided and conversation summary guided training data is not combined, techniques described above relative to a conversation summary guided synthetic doctor-patient conversation generation process can nonetheless be applied to a knowledge graph guided synthetic doctor-patient conversation generation process. For example, it is possible to apply the back translation augmentation and/or filler word redistribution techniques described with respect to a conversation summary guided synthetic doctor-patient conversation generation process to a knowledge graph guided synthetic doctor-patient conversation generation process to improve the language diversity thereof.

### D. Use of Generated Synthetic Doctor-Patient Conversation Data

Synthetic doctor-patient conversations generated according to a knowledge graph guided synthetic doctor-patient conversation generation process, a conversation summary guided synthetic doctor-patient conversation generation process, or a combined knowledge graph and conversation summary guided synthetic doctor-patient conversation generation process can be used for various purposes. For example, in one use case, synthetic doctor-patient conversation data generated by a knowledge graph guided synthetic doctor-patient conversation process may be used to train a model to identify and redact personal health information (PHI) or personal identifiable information (PII) from text. In another example, synthetic doctor-patient conversation data generated by a conversation summary guided synthetic doctor-patient conversation generation process may be used to recreate doctor-patient conversations from summaries such as clinician SOAP notes. Generated synthetic doctor-patient conversation data can also be used to improve the automatic speech recognition (ASR) capabilities of a model, such as may be utilized in a chatbot system. Other uses are also possible, and nothing herein is to be interpreted as limiting the use of generated synthetic doctor-patient conversation data to a particular purpose.

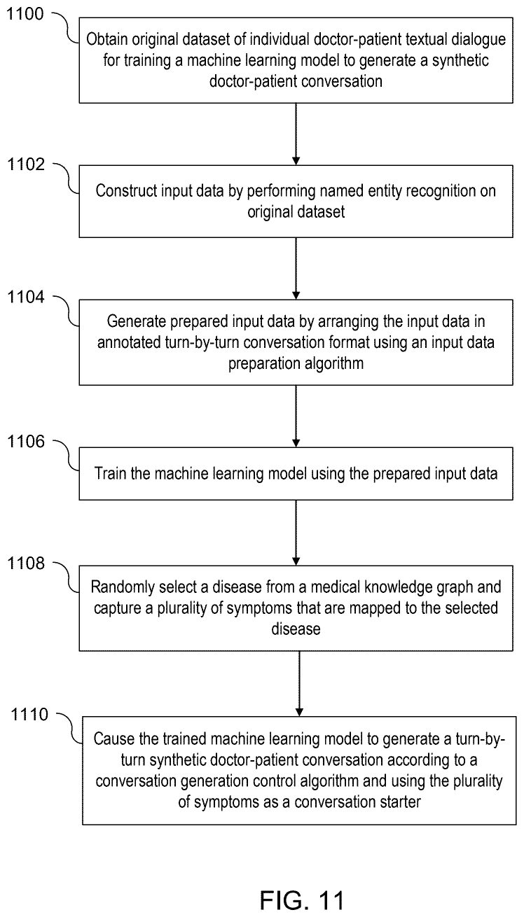

One example of a knowledge graph guided method for training a machine learning model to generate a synthetic doctor-patient conversation is represented in the flowchart of FIG. 11. As indicated at block 1100, an original dataset can be obtained for the purpose of training a machine learning model to generate a synthetic doctor-patient conversation. The original dataset may include data in the form of textual dialogue associated with each of a plurality of individual doctor-patient conversations. The original dataset may be a dataset comprising curated doctor-patient conversation data, In an example, the original dataset can be a combination of the PriMock57 dataset and the Nature dataset. The data of the original dataset may be split into a training dataset and a test dataset.

Input data may next be constructed, as indicated at block 1102. The input data may be constructed by performing a named entity recognition (NER) operation on the dialogue of the original dataset that captures and categorizes named medical entities present in the dialogue. The NER may utilize one or more pre-trained models configured to collectively identify and classify biomedical, PHI and medical entities in text. The NER operation can be performed on the dialogue of the original dataset in a turn-by-turn fashion. The NER operation may be followed by a medical NER operation, whereby one or more pre-trained models can be used to break down the dialogue of the original dataset into simpler, smaller sentences at a conversation turn level and to detect medical-specific entities within the conversation turns.

According to block 1104, prepared input data may be generated after the input data has been constructed by arranging the input data in an annotated turn-by-turn conversation format using an input data preparation algorithm having various control parameters. The control parameters can include, but are not necessarily limited to, symptoms/topics, entities sampled from the symptoms/topics, speaker identity, conversation turn length, remaining conversation turns, and context. The context control parameter for a given conversation turn may comprise the cumulative dialogue of all the preceding conversation turns, when there is at least one preceding conversation turn. Symptoms identification and keyword extraction operations may also be performed.

The machine learning model can then be trained using the prepared input data, as represented at block 1106. Training the machine learning model can include multistage finetuning of the machine learning model. In some embodiments, multistage finetuning may include first-fold finetuning using a medical question answering dataset, followed by second-fold finetuning. Second-fold finetuning may be guided by the input data preparation algorithm and the control parameters thereof and can use a dataset including data in the form of textual dialogue associated with each of a plurality of individual non-medical specific conversations. Second-fold finetuning may be followed by final-fold finetuning. Final-fold finetuning may also be guided by the input data preparation algorithm and the control parameters thereof and can use a dataset including data in the form of textual dialogue associated with each of a plurality of individual medical-specific conversations. The symptoms/topics control parameter of the input data preparation algorithm may be composed of keywords previously derived from a dataset including data in the form of textual dialogue associated with each of a plurality of individual non-medical conversations.

For purposes of using the trained machine learning model to generate a synthetic doctor-patient conversation, a disease can be randomly selected from a medical knowledge graph and a plurality of symptoms that are mapped to the selected disease may be captured, as indicated in block 1108. The captured symptoms can be used a seed data to cause the trained machine learning model to begin generating a synthetic doctor-patient conversation, as represented in block 1110. More specifically, the trained machine learning model can be caused to generate a turn-by-turn synthetic doctor-patient conversation according to a conversation generation control algorithm having various control parameters, by inputting the plurality of symptoms to the machine learning model as a first control parameter of the conversation generation control algorithm and randomly assigning a first speaker. Performance of the trained machine learning model can be evaluated by analyzing the generated turn-by-turn synthetic doctor-patient conversation using various techniques. For example, trained machine learning model performance can be evaluated by analyzing the generated turn-by-turn synthetic doctor-patient conversation using a metric such as recall-oriented understudy for gisting evaluation, n-gram diversity score, unique n-gram count, and combinations thereof.

### Illustrative Systems

Infrastructure as a service (IaaS) is one particular type of cloud computing that can be used to implement the various techniques described herein for generating synthetic doctor-patient conversations using medical knowledge graphs and/or doctor-patient conversation summaries to guide and control the synthetic doctor-patient conversation generation process. IaaS can be configured to provide virtualized computing resources over a public network (e.g., the Internet). In an IaaS model, a cloud computing provider can host the infrastructure components (e.g., servers, storage devices, network nodes (e.g., hardware), deployment software, platform virtualization (e.g., a hypervisor layer), or the like). In some cases, an IaaS provider may also supply a variety of services to accompany those infrastructure components (example services include billing software, monitoring software, logging software, load balancing software, clustering software, etc.). Thus, as these services may be policy-driven, IaaS users may be able to implement policies to drive load balancing to maintain application availability and performance.

In some instances, IaaS customers may access resources and services through a wide area network (WAN), such as the Internet, and can use the cloud provider's services to install the remaining elements of an application stack. For example, the user can log in to the IaaS platform to create virtual machines (VMs), install operating systems (OSs) on each VM, deploy middleware such as databases, create storage buckets for workloads and backups, and even install enterprise software into that VM. Customers can then use the provider's services to perform various functions, including balancing network traffic, troubleshooting application issues, monitoring performance, managing disaster recovery, etc.

In most cases, a cloud computing model will require the participation of a cloud provider. The cloud provider may, but need not be, a third-party service that specializes in providing (e.g., offering, renting, selling) IaaS. An entity might also opt to deploy a private cloud, becoming its own provider of infrastructure services.

In some examples, IaaS deployment is the process of putting a new application, or a new version of an application, onto a prepared application server or the like. It may also include the process of preparing the server (e.g., installing libraries, daemons, etc.). This is often managed by the cloud provider, below the hypervisor layer (e.g., the servers, storage, network hardware, and virtualization). Thus, the customer may be responsible for handling (OS), middleware, and/or application deployment (e.g., on self-service virtual machines (e.g., that can be spun up on demand) or the like.

In some examples, IaaS provisioning may refer to acquiring computers or virtual hosts for use, and even installing needed libraries or services on them. In most cases, deployment does not include provisioning, and the provisioning may need to be performed first.

In some cases, there are two different challenges for IaaS provisioning. First, there is the initial challenge of provisioning the initial set of infrastructure before anything is running. Second, there is the challenge of evolving the existing infrastructure (e.g., adding new services, changing services, removing services, etc.) once everything has been provisioned. In some cases, these two challenges may be addressed by enabling the configuration of the infrastructure to be defined declaratively. In other words, the infrastructure (e.g., what components are needed and how they interact) can be defined by one or more configuration files. Thus, the overall topology of the infrastructure (e.g., what resources depend on which, and how they each work together) can be described declaratively. In some instances, once the topology is defined, a workflow can be generated that creates and/or manages the different components described in the configuration files.

In some examples, an infrastructure may have many interconnected elements. For example, there may be one or more virtual private clouds (VPCs) (e.g., a potentially on-demand pool of configurable and/or shared computing resources), also known as a core network. In some examples, there may also be one or more inbound/outbound traffic group rules provisioned to define how the inbound and/or outbound traffic of the network will be set up and one or more virtual machines (VMs). Other infrastructure elements may also be provisioned, such as a load balancer, a database, or the like. As more and more infrastructure elements are desired and/or added, the infrastructure may incrementally evolve.

In some instances, continuous deployment techniques may be employed to enable deployment of infrastructure code across various virtual computing environments. Additionally, the described techniques can enable infrastructure management within these environments. In some examples, service teams can write code that is desired to be deployed to one or more, but often many, different production environments (e.g., across various different geographic locations, sometimes spanning the entire world). However, in some examples, the infrastructure on which the code will be deployed must first be set up. In some instances, the provisioning can be done manually, a provisioning tool may be utilized to provision the resources, and/or deployment tools may be utilized to deploy the code once the infrastructure is provisioned.

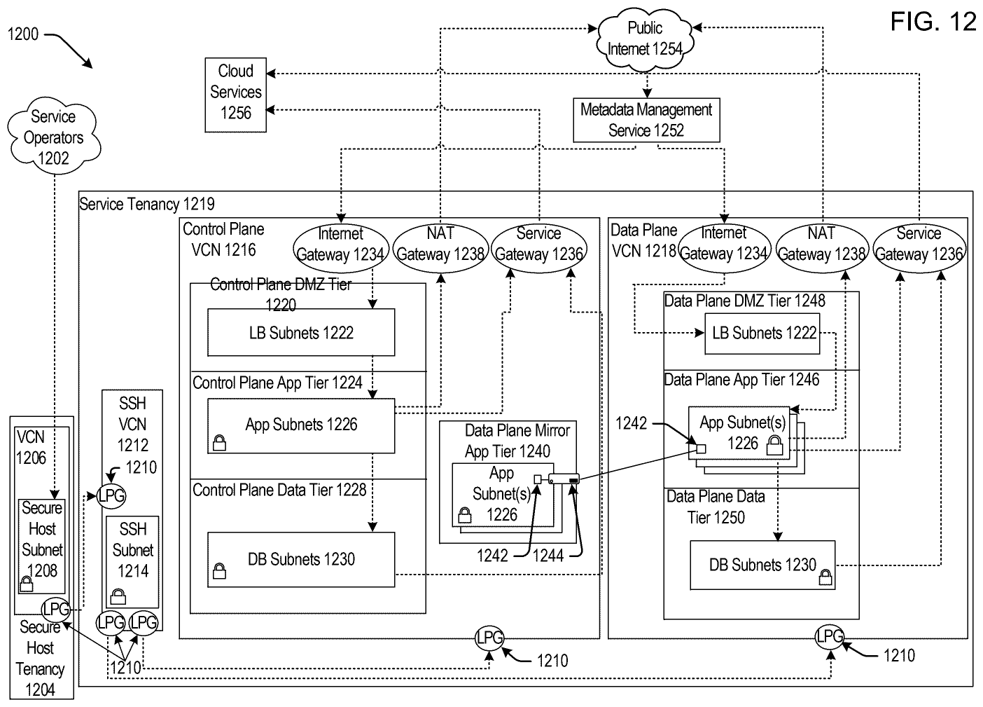

FIG. 12 is a block diagram 1200 illustrating an example pattern of an IaaS architecture, according to at least one embodiment. Service operators 1202 can be communicatively coupled to a secure host tenancy 1204 that can include a virtual cloud network (VCN) 1206 and a secure host subnet 1208. In some examples, the service operators 1202 may be using one or more client computing devices, which may be portable handheld devices (e.g., an iPhone®, cellular telephone, an iPad®, computing tablet, a personal digital assistant (PDA)) or wearable devices (e.g., a Google Glass® head mounted display), running software such as Microsoft Windows Mobile®, and/or a variety of mobile operating systems such as iOS, Windows Phone, Android, BlackBerry 8, Palm OS, and the like, and being Internet, e-mail, short message service (SMS), Blackberry®, or other communication protocol enabled. Alternatively, the client computing devices can be general purpose personal computers including, by way of example, personal computers and/or laptop computers running various versions of Microsoft Windows®, Apple Macintosh®, and/or Linux operating systems. The client computing devices can be workstation computers running any of a variety of commercially-available UNIX® or UNIX-like operating systems, including without limitation the variety of GNU/Linux operating systems, such as for example, Google Chrome OS. Alternatively, or in addition, client computing devices may be any other electronic device, such as a thin-client computer, an Internet-enabled gaming system (e.g., a Microsoft Xbox gaming console with or without a Kinect® gesture input device), and/or a personal messaging device, capable of communicating over a network that can access the VCN 1206 and/or the Internet.

The VCN 1206 can include a local peering gateway (LPG) 1210 that can be communicatively coupled to a secure shell (SSH) VCN 1212 via an LPG 1210 contained in the SSH VCN 1212. The SSH VCN 1212 can include an SSH subnet 1214, and the SSH VCN 1212 can be communicatively coupled to a control plane VCN 1216 via the LPG 1210 contained in the control plane VCN 1216. Also, the SSH VCN 1212 can be communicatively coupled to a data plane VCN 1218 via an LPG 1210. The control plane VCN 1216 and the data plane VCN 1218 can be contained in a service tenancy 1219 that can be owned and/or operated by the IaaS provider.

The control plane VCN 1216 can include a control plane demilitarized zone (DMZ) tier 1220 that acts as a perimeter network (e.g., portions of a corporate network between the corporate intranet and external networks). The DMZ-based servers may have restricted responsibilities and help keep breaches contained. Additionally, the DMZ tier 1220 can include one or more load balancer (LB) subnet(s) 1222, a control plane app tier 1224 that can include app subnet(s) 1226, a control plane data tier 1228 that can include database (DB) subnet(s) 1230 (e.g., frontend DB subnet(s) and/or backend DB subnet(s)). The LB subnet(s) 1222 contained in the control plane DMZ tier 1220 can be communicatively coupled to the app subnet(s) 1226 contained in the control plane app tier 1224 and an Internet gateway 1234 that can be contained in the control plane VCN 1216, and the app subnet(s) 1226 can be communicatively coupled to the DB subnet(s) 1230 contained in the control plane data tier 1228 and a service gateway 1236 and a network address translation (NAT) gateway 12312. The control plane VCN 1216 can include the service gateway 1236 and the NAT gateway 1238.

The control plane VCN 1216 can include a data plane mirror app tier 1240 that can include app subnet(s) 1226. The app subnet(s) 1226 contained in the data plane mirror app tier 1240 can include a virtual network interface controller (VNIC) 1242 that can execute a compute instance 1244. The compute instance 1244 can communicatively couple the app subnet(s) 1226 of the data plane mirror app tier 1240 to app subnet(s) 1226 that can be contained in a data plane app tier 1246.

The data plane VCN 1218 can include the data plane app tier 1246, a data plane DMZ tier 1248, and a data plane data tier 1250. The data plane DMZ tier 1248 can include LB subnet(s) 1222 that can be communicatively coupled to the app subnet(s) 1226 of the data plane app tier 1246 and the Internet gateway 1234 of the data plane VCN 1218. The app subnet(s) 1226 can be communicatively coupled to the service gateway 1236 of the data plane VCN 1218 and the NAT gateway 1238 of the data plane VCN 1218. The data plane data tier 1250 can also include the DB subnet(s) 1230 that can be communicatively coupled to the app subnet(s) 1226 of the data plane app tier 1246.

The Internet gateway 1234 of the control plane VCN 1216 and of the data plane VCN 1218 can be communicatively coupled to a metadata management service 1252 that can be communicatively coupled to public Internet 1254. Public Internet 1254 can be communicatively coupled to the NAT gateway 1238 of the control plane VCN 1216 and of the data plane VCN 1218. The service gateway 1236 of the control plane VCN 1216 and of the data plane VCN 1218 can be communicatively coupled to cloud services 1256.

In some examples, the service gateway 1236 of the control plane VCN 1216 or of the data plane VCN 1218 can make application programming interface (API) calls to cloud services 1256 without going through public Internet 1254. The API calls to cloud services 1256 from the service gateway 1236 can be one-way: the service gateway 1236 can make API calls to cloud services 1256, and cloud services 1256 can send requested data to the service gateway 1236. But cloud services 1256 may not initiate API calls to the service gateway 1236.

In some examples, the secure host tenancy 1204 can be directly connected to the service tenancy 1219, which may be otherwise isolated. The secure host subnet 1208 can communicate with the SSH subnet 1214 through an LPG 1210 that may enable two-way communication over an otherwise isolated system. Connecting the secure host subnet 1208 to the SSH subnet 1214 may give the secure host subnet 1208 access to other entities within the service tenancy 1219.

The control plane VCN 1216 may allow users of the service tenancy 1219 to set up or otherwise provision desired resources. Desired resources provisioned in the control plane VCN 1216 may be deployed or otherwise used in the data plane VCN 1218. In some examples, the control plane VCN 1216 can be isolated from the data plane VCN 1218, and the data plane mirror app tier 1240 of the control plane VCN 1216 can communicate with the data plane app tier 1246 of the data plane VCN 1218 via VNICs 1242 that can be contained in the data plane mirror app tier 1240 and the data plane app tier 1246.

In some examples, users of the system, or customers, can make requests, for example create, read, update, or delete (CRUD) operations, through public Internet 1254 that can communicate the requests to the metadata management service 1252. The metadata management service 1252 can communicate the request to the control plane VCN 1216 through the Internet gateway 1234. The request can be received by the LB subnet(s) 1222 contained in the control plane DMZ tier 1220. The LB subnet(s) 1222 may determine that the request is valid, and in response to this determination, the LB subnet(s) 1222 can transmit the request to app subnet(s) 1226 contained in the control plane app tier 1224. If the request is validated and requires a call to public Internet 1254, the call to public Internet 1254 may be transmitted to the NAT gateway 1238 that can make the call to public Internet 1254. Metadata that may be desired to be stored by the request can be stored in the DB subnet(s) 1230.

In some examples, the data plane mirror app tier 1240 can facilitate direct communication between the control plane VCN 1216 and the data plane VCN 1218. For example, changes, updates, or other suitable modifications to configuration may be desired to be applied to the resources contained in the data plane VCN 1218. Via a VNIC 1242, the control plane VCN 1216 can directly communicate with, and can thereby execute the changes, updates, or other suitable modifications to configuration to, resources contained in the data plane VCN 1218.

In some embodiments, the control plane VCN 1216 and the data plane VCN 1218 can be contained in the service tenancy 1219. In this case, the user, or the customer, of the system may not own or operate either the control plane VCN 1216 or the data plane VCN 1218. Instead, the IaaS provider may own or operate the control plane VCN 1216 and the data plane VCN 1218, both of which may be contained in the service tenancy 1219. This embodiment can enable isolation of networks that may prevent users or customers from interacting with other users', or other customers', resources. Also, this embodiment may allow users or customers of the system to store databases privately without needing to rely on public Internet 1254, which may not have a desired level of threat prevention, for storage.

In other embodiments, the LB subnet(s) 1222 contained in the control plane VCN 1216 can be configured to receive a signal from the service gateway 1236. In this embodiment, the control plane VCN 1216 and the data plane VCN 1218 may be configured to be called by a customer of the IaaS provider without calling public Internet 1254. Customers of the IaaS provider may desire this embodiment since database(s) that the customers use may be controlled by the IaaS provider and may be stored on the service tenancy 1219, which may be isolated from public Internet 1254.

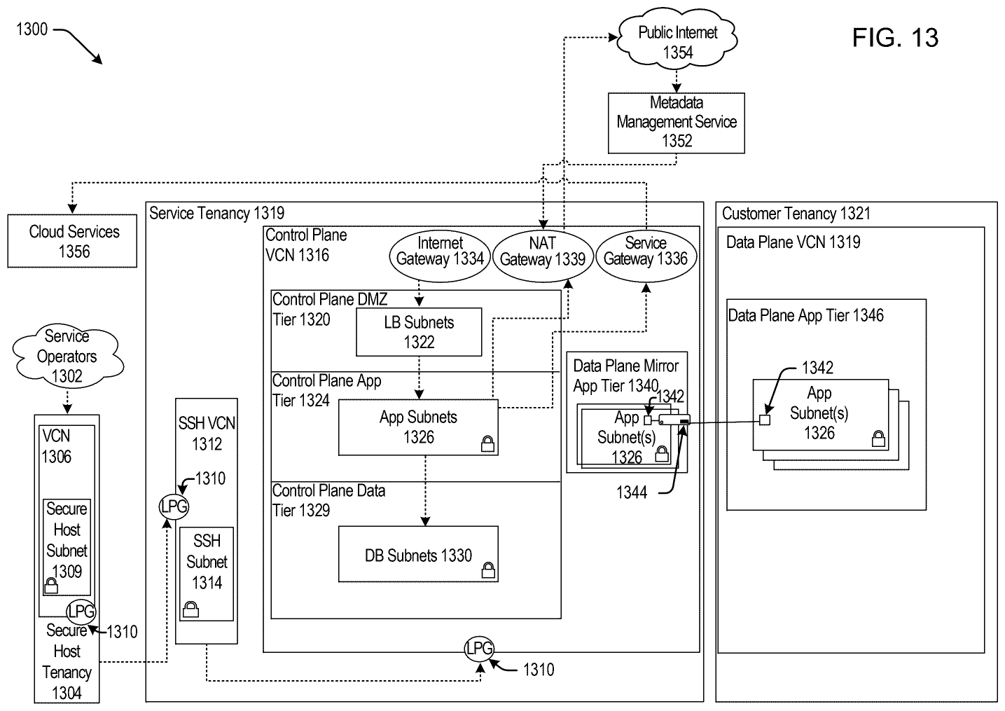

FIG. 13 is a block diagram 1300 illustrating another example pattern of an IaaS architecture, according to at least one embodiment. Service operators 1302 (e.g., service operators 1202 of FIG. 12) can be communicatively coupled to a secure host tenancy 1304 (e.g., the secure host tenancy 1204 of FIG. 12) that can include a virtual cloud network (VCN) 1306 (e.g., the VCN 1206 of FIG. 12) and a secure host subnet 1308 (e.g., the secure host subnet 1208 of FIG. 12). The VCN 1306 can include a local peering gateway (LPG) 1310 (e.g., the LPG 1210 of FIG. 12) that can be communicatively coupled to a secure shell (SSH) VCN 1312 (e.g., the SSH VCN 1212 of FIG. 12) via an LPG 1310 contained in the SSH VCN 1312. The SSH VCN 1312 can include an SSH subnet 1314 (e.g., the SSH subnet 1214 of FIG. 12), and the SSH VCN 1312 can be communicatively coupled to a control plane VCN 1316 (e.g., the control plane VCN 1216 of FIG. 12) via an LPG 1310 contained in the control plane VCN 1316. The control plane VCN 1316 can be contained in a service tenancy 1319 (e.g., the service tenancy 1219 of FIG. 12), and the data plane VCN 1318 (e.g., the data plane VCN 1218 of FIG. 12) can be contained in a customer tenancy 1321 that may be owned or operated by users, or customers, of the system.

The control plane VCN 1316 can include a control plane DMZ tier 1320 (e.g., the control plane DMZ tier 1220 of FIG. 12) that can include LB subnet(s) 1322 (e.g., LB subnet(s) 1222 of FIG. 12), a control plane app tier 1324 (e.g., the control plane app tier 1224 of FIG. 12) that can include app subnet(s) 1326 (e.g., app subnet(s) 1226 of FIG. 12), a control plane data tier 1328 (e.g., the control plane data tier 1228 of FIG. 12) that can include database (DB) subnet(s) 1330 (e.g., similar to DB subnet(s) 1230 of FIG. 12). The LB subnet(s) 1322 contained in the control plane DMZ tier 1320 can be communicatively coupled to the app subnet(s) 1326 contained in the control plane app tier 1324 and an Internet gateway 1334 (e.g., the Internet gateway 1234 of FIG. 12) that can be contained in the control plane VCN 1316, and the app subnet(s) 1326 can be communicatively coupled to the DB subnet(s) 1330 contained in the control plane data tier 1328 and a service gateway 1336 (e.g., the service gateway 1236 of FIG. 12) and a network address translation (NAT) gateway 1338 (e.g., the NAT gateway 1238 of FIG. 12). The control plane VCN 1316 can include the service gateway 1336 and the NAT gateway 1338.

The control plane VCN 1316 can include a data plane mirror app tier 1340 (e.g., the data plane mirror app tier 1240 of FIG. 12) that can include app subnet(s) 1326. The app subnet(s) 1326 contained in the data plane mirror app tier 1340 can include a virtual network interface controller (VNIC) 1342 (e.g., the VNIC of 1242) that can execute a compute instance 1344 (e.g., similar to the compute instance 1244 of FIG. 12). The compute instance 1344 can facilitate communication between the app subnet(s) 1326 of the data plane mirror app tier 1340 and the app subnet(s) 1326 that can be contained in a data plane app tier 1346 (e.g., the data plane app tier 1246 of FIG. 12) via the VNIC 1342 contained in the data plane mirror app tier 1340 and the VNIC 1342 contained in the data plane app tier 1346.

The Internet gateway 1334 contained in the control plane VCN 1316 can be communicatively coupled to a metadata management service 1352 (e.g., the metadata management service 1252 of FIG. 12) that can be communicatively coupled to public Internet 1354 (e.g., public Internet 1254 of FIG. 12). Public Internet 1354 can be communicatively coupled to the NAT gateway 1338 contained in the control plane VCN 1316. The service gateway 1336 contained in the control plane VCN 1316 can be communicatively coupled to cloud services 1356 (e.g., cloud services 1256 of FIG. 12).

In some examples, the data plane VCN 1318 can be contained in the customer tenancy 1321. In this case, the IaaS provider may provide the control plane VCN 1316 for each customer, and the IaaS provider may, for each customer, set up a unique compute instance 1344 that is contained in the service tenancy 1319. Each compute instance 1344 may allow communication between the control plane VCN 1316, contained in the service tenancy 1319, and the data plane VCN 1318 that is contained in the customer tenancy 1321. The compute instance 1344 may allow resources, that are provisioned in the control plane VCN 1316 that is contained in the service tenancy 1319, to be deployed or otherwise used in the data plane VCN 1318 that is contained in the customer tenancy 1321.

In other examples, the customer of the IaaS provider may have databases that live in the customer tenancy 1321. In this example, the control plane VCN 1316 can include the data plane mirror app tier 1340 that can include app subnet(s) 1326. The data plane mirror app tier 1340 can reside in the data plane VCN 1318, but the data plane mirror app tier 1340 may not live in the data plane VCN 1318. That is, the data plane mirror app tier 1340 may have access to the customer tenancy 1321, but the data plane mirror app tier 1340 may not exist in the data plane VCN 1318 or be owned or operated by the customer of the IaaS provider. The data plane mirror app tier 1340 may be configured to make calls to the data plane VCN 1318 but may not be configured to make calls to any entity contained in the control plane VCN 1316. The customer may desire to deploy or otherwise use resources in the data plane VCN 1318 that are provisioned in the control plane VCN 1316, and the data plane mirror app tier 1340 can facilitate the desired deployment, or other usage of resources, of the customer.

In some embodiments, the customer of the IaaS provider can apply filters to the data plane VCN 1318. In this embodiment, the customer can determine what the data plane VCN 1318 can access, and the customer may restrict access to public Internet 1354 from the data plane VCN 1318. The IaaS provider may not be able to apply filters or otherwise control access of the data plane VCN 1318 to any outside networks or databases. Applying filters and controls by the customer onto the data plane VCN 1318, contained in the customer tenancy 1321, can help isolate the data plane VCN 1318 from other customers and from public Internet 1354.

In some embodiments, cloud services 1356 can be called by the service gateway 1336 to access services that may not exist on public Internet 1354, on the control plane VCN 1316, or on the data plane VCN 1318. The connection between cloud services 1356 and the control plane VCN 1316 or the data plane VCN 1318 may not be live or continuous. Cloud services 1356 may exist on a different network owned or operated by the IaaS provider. Cloud services 1356 may be configured to receive calls from the service gateway 1336 and may be configured to not receive calls from public Internet 1354. Some cloud services 1356 may be isolated from other cloud services 1356, and the control plane VCN 1316 may be isolated from cloud services 1356 that may not be in the same region as the control plane VCN 1316. For example, the control plane VCN 1316 may be located in “Region 1,” and cloud service “Deployment 12,” may be located in Region 1 and in “Region 2.” If a call to Deployment 12 is made by the service gateway 1336 contained in the control plane VCN 1316 located in Region 1, the call may be transmitted to Deployment 12 in Region 1. In this example, the control plane VCN 1316, or Deployment 12 in Region 1, may not be communicatively coupled to, or otherwise in communication with, Deployment 12 in Region 2.

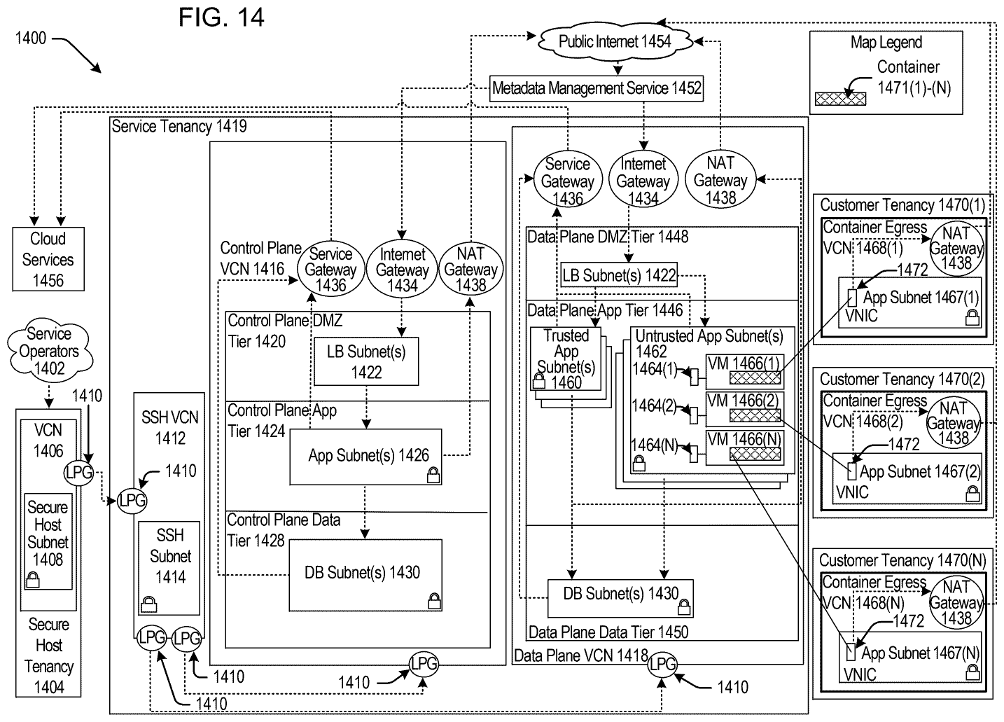

FIG. 14 is a block diagram 1400 illustrating another example pattern of an IaaS architecture, according to at least one embodiment. Service operators 1402 (e.g., service operators 1202 of FIG. 12) can be communicatively coupled to a secure host tenancy 1404 (e.g., the secure host tenancy 1204 of FIG. 12) that can include a virtual cloud network (VCN) 1406 (e.g., the VCN 1206 of FIG. 12) and a secure host subnet 1408 (e.g., the secure host subnet 1208 of FIG. 12). The VCN 1406 can include an LPG 1410 (e.g., the LPG 1210 of FIG. 12) that can be communicatively coupled to an SSH VCN 1412 (e.g., the SSH VCN 1212 of FIG. 12) via an LPG 1410 contained in the SSH VCN 1412. The SSH VCN 1412 can include an SSH subnet 1414 (e.g., the SSH subnet 1214 of FIG. 12), and the SSH VCN 1412 can be communicatively coupled to a control plane VCN 1416 (e.g., the control plane VCN 1216 of FIG. 12) via an LPG 1410 contained in the control plane VCN 1416 and to a data plane VCN 1418 (e.g., the data plane 1218 of FIG. 12) via an LPG 1410 contained in the data plane VCN 1418. The control plane VCN 1416 and the data plane VCN 1418 can be contained in a service tenancy 1419 (e.g., the service tenancy 1219 of FIG. 12).

The control plane VCN 1416 can include a control plane DMZ tier 1420 (e.g., the control plane DMZ tier 1220 of FIG. 12) that can include load balancer (LB) subnet(s) 1422 (e.g., LB subnet(s) 1222 of FIG. 12), a control plane app tier 1424 (e.g., the control plane app tier 1224 of FIG. 12) that can include app subnet(s) 1426 (e.g., similar to app subnet(s) 1226 of FIG. 12), a control plane data tier 1428 (e.g., the control plane data tier 1228 of FIG. 12) that can include DB subnet(s) 1430. The LB subnet(s) 1422 contained in the control plane DMZ tier 1420 can be communicatively coupled to the app subnet(s) 1426 contained in the control plane app tier 1424 and to an Internet gateway 1434 (e.g., the Internet gateway 1234 of FIG. 12) that can be contained in the control plane VCN 1416, and the app subnet(s) 1426 can be communicatively coupled to the DB subnet(s) 1430 contained in the control plane data tier 1428 and to a service gateway 1436 (e.g., the service gateway of FIG. 12) and a network address translation (NAT) gateway 1438 (e.g., the NAT gateway 1238 of FIG. 12). The control plane VCN 1416 can include the service gateway 1436 and the NAT gateway 1438.

The data plane VCN 1418 can include a data plane app tier 1446 (e.g., the data plane app tier 1246 of FIG. 12), a data plane DMZ tier 1448 (e.g., the data plane DMZ tier 1248 of FIG. 12), and a data plane data tier 1450 (e.g., the data plane data tier 1250 of FIG. 12). The data plane DMZ tier 1448 can include LB subnet(s) 1422 that can be communicatively coupled to trusted app subnet(s) 1460 and untrusted app subnet(s) 1462 of the data plane app tier 1446 and the Internet gateway 1434 contained in the data plane VCN 1418. The trusted app subnet(s) 1460 can be communicatively coupled to the service gateway 1436 contained in the data plane VCN 1418, the NAT gateway 1438 contained in the data plane VCN 1418, and DB subnet(s) 1430 contained in the data plane data tier 1450. The untrusted app subnet(s) 1462 can be communicatively coupled to the service gateway 1436 contained in the data plane VCN 1418 and DB subnet(s) 1430 contained in the data plane data tier 1450. The data plane data tier 1450 can include DB subnet(s) 1430 that can be communicatively coupled to the service gateway 1436 contained in the data plane VCN 1418.

The untrusted app subnet(s) 1462 can include one or more primary VNICs 1464(1)-(N) that can be communicatively coupled to tenant virtual machines (VMs) 1466(1)-(N). Each tenant VM 1466(1)-(N) can be communicatively coupled to a respective app subnet 1467(1)-(N) that can be contained in respective container egress VCNs 1468(1)-(N) that can be contained in respective customer tenancies 1470(1)-(N). Respective secondary VNICs 1472(1)-(N) can facilitate communication between the untrusted app subnet(s) 1462 contained in the data plane VCN 1418 and the app subnet contained in the container egress VCNs 1468(1)-(N). Each container egress VCNs 1468(1)-(N) can include a NAT gateway 1438 that can be communicatively coupled to public Internet 1454 (e.g., public Internet 1254 of FIG. 12).

The Internet gateway 1434 contained in the control plane VCN 1416 and contained in the data plane VCN 1418 can be communicatively coupled to a metadata management service 1452 (e.g., the metadata management system 1252 of FIG. 12) that can be communicatively coupled to public Internet 1454. Public Internet 1454 can be communicatively coupled to the NAT gateway 1438 contained in the control plane VCN 1416 and contained in the data plane VCN 1418. The service gateway 1436 contained in the control plane VCN 1416 and contained in the data plane VCN 1418 can be communicatively coupled to cloud services 1456.

In some embodiments, the data plane VCN 1418 can be integrated with customer tenancies 1470. This integration can be useful or desirable for customers of the IaaS provider in some cases such as a case that may desire support when executing code. The customer may provide code to run that may be destructive, may communicate with other customer resources, or may otherwise cause undesirable effects. In response to this, the IaaS provider may determine whether to run code given to the IaaS provider by the customer.

In some examples, the customer of the IaaS provider may grant temporary network access to the IaaS provider and request a function to be attached to the data plane app tier 1446. Code to run the function may be executed in the VMs 1466(1)-(N), and the code may not be configured to run anywhere else on the data plane VCN 1418. Each VM 1466(1)-(N) may be connected to one customer tenancy 1470. Respective containers 1471(1)-(N) contained in the VMs 1466(1)-(N) may be configured to run the code. In this case, there can be a dual isolation (e.g., the containers 1471(1)-(N) running code, where the containers 1471(1)-(N) may be contained in at least the VM 1466(1)-(N) that are contained in the untrusted app subnet(s) 1462), which may help prevent incorrect or otherwise undesirable code from damaging the network of the IaaS provider or from damaging a network of a different customer. The containers 1471(1)-(N) may be communicatively coupled to the customer tenancy 1470 and may be configured to transmit or receive data from the customer tenancy 1470. The containers 1471(1)-(N) may not be configured to transmit or receive data from any other entity in the data plane VCN 1418. Upon completion of running the code, the IaaS provider may kill or otherwise dispose of the containers 1471(1)-(N).

In some embodiments, the trusted app subnet(s) 1460 may run code that may be owned or operated by the IaaS provider. In this embodiment, the trusted app subnet(s) 1460 may be communicatively coupled to the DB subnet(s) 1430 and be configured to execute CRUD operations in the DB subnet(s) 1430. The untrusted app subnet(s) 1462 may be communicatively coupled to the DB subnet(s) 1430, but in this embodiment, the untrusted app subnet(s) may be configured to execute read operations in the DB subnet(s) 1430. The containers 1471(1)-(N) that can be contained in the VM 1466(1)-(N) of each customer and that may run code from the customer may not be communicatively coupled with the DB subnet(s) 1430.

In other embodiments, the control plane VCN 1416 and the data plane VCN 1418 may not be directly communicatively coupled. In this embodiment, there may be no direct communication between the control plane VCN 1416 and the data plane VCN 1418. However, communication can occur indirectly through at least one method. An LPG 1410 may be established by the IaaS provider that can facilitate communication between the control plane VCN 1416 and the data plane VCN 1418. In another example, the control plane VCN 1416 or the data plane VCN 1418 can make a call to cloud services 1456 via the service gateway 1436. For example, a call to cloud services 1456 from the control plane VCN 1416 can include a request for a service that can communicate with the data plane VCN 1418.

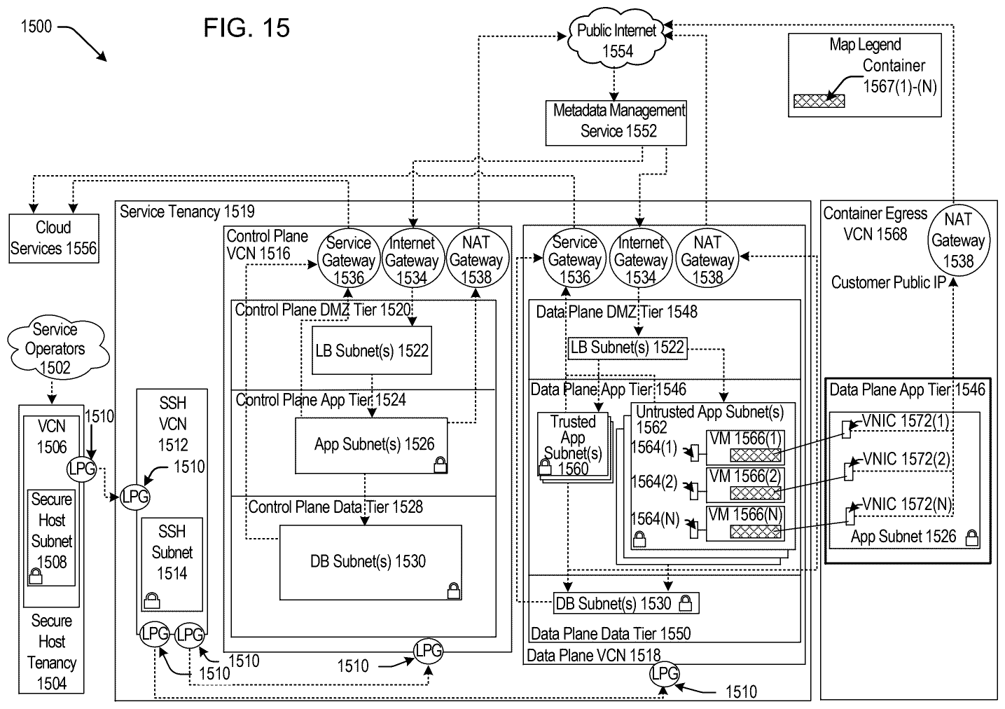

FIG. 15 is a block diagram 1500 illustrating another example pattern of an IaaS architecture, according to at least one embodiment. Service operators 1502 (e.g., service operators 1202 of FIG. 12) can be communicatively coupled to a secure host tenancy 1504 (e.g., the secure host tenancy 1204 of FIG. 12) that can include a virtual cloud network (VCN) 1506 (e.g., the VCN 1206 of FIG. 12) and a secure host subnet 1508 (e.g., the secure host subnet 1208 of FIG. 12). The VCN 1506 can include an LPG 1510 (e.g., the LPG 1210 of FIG. 12) that can be communicatively coupled to an SSH VCN 1512 (e.g., the SSH VCN 1212 of FIG. 12) via an LPG 1510 contained in the SSH VCN 1512. The SSH VCN 1512 can include an SSH subnet 1514 (e.g., the SSH subnet 1214 of FIG. 12), and the SSH VCN 1512 can be communicatively coupled to a control plane VCN 1516 (e.g., the control plane VCN 1216 of FIG. 12) via an LPG 1510 contained in the control plane VCN 1516 and to a data plane VCN 1518 (e.g., the data plane 1218 of FIG. 12) via an LPG 1510 contained in the data plane VCN 1518. The control plane VCN 1516 and the data plane VCN 1518 can be contained in a service tenancy 1519 (e.g., the service tenancy 1219 of FIG. 12).

The control plane VCN 1516 can include a control plane DMZ tier 1520 (e.g., the control plane DMZ tier 1220 of FIG. 12) that can include LB subnet(s) 1522 (e.g., LB subnet(s) 1222 of FIG. 12), a control plane app tier 1524 (e.g., the control plane app tier 1224 of FIG. 12) that can include app subnet(s) 1526 (e.g., app subnet(s) 1226 of FIG. 12), a control plane data tier 1528 (e.g., the control plane data tier 1228 of FIG. 12) that can include DB subnet(s) 1530 (e.g., DB subnet(s) 930 of FIG. 14). The LB subnet(s) 1522 contained in the control plane DMZ tier 1520 can be communicatively coupled to the app subnet(s) 1526 contained in the control plane app tier 1524 and to an Internet gateway 1534 (e.g., the Internet gateway 1234 of FIG. 12) that can be contained in the control plane VCN 1516, and the app subnet(s) 1526 can be communicatively coupled to the DB subnet(s) 1530 contained in the control plane data tier 1528 and to a service gateway 1536 (e.g., the service gateway of FIG. 12) and a network address translation (NAT) gateway 1538 (e.g., the NAT gateway 1238 of FIG. 12). The control plane VCN 1516 can include the service gateway 1536 and the NAT gateway 1538.

The data plane VCN 1518 can include a data plane app tier 1546 (e.g., the data plane app tier 1246 of FIG. 12), a data plane DMZ tier 1548 (e.g., the data plane DMZ tier 1248 of FIG. 12), and a data plane data tier 1550 (e.g., the data plane data tier 1250 of FIG. 12). The data plane DMZ tier 1548 can include LB subnet(s) 1522 that can be communicatively coupled to trusted app subnet(s) 1560 (e.g., trusted app subnet(s) 1460 of FIG. 14) and untrusted app subnet(s) 1562 (e.g., untrusted app subnet(s) 1462 of FIG. 14) of the data plane app tier 1546 and the Internet gateway 1534 contained in the data plane VCN 1518. The trusted app subnet(s) 1560 can be communicatively coupled to the service gateway 1536 contained in the data plane VCN 1518, the NAT gateway 1538 contained in the data plane VCN 1518, and DB subnet(s) 1530 contained in the data plane data tier 1550. The untrusted app subnet(s) 1562 can be communicatively coupled to the service gateway 1536 contained in the data plane VCN 1518 and DB subnet(s) 1530 contained in the data plane data tier 1550. The data plane data tier 1550 can include DB subnet(s) 1530 that can be communicatively coupled to the service gateway 1536 contained in the data plane VCN 1518.

The untrusted app subnet(s) 1562 can include primary VNICs 1564(1)-(N) that can be communicatively coupled to tenant virtual machines (VMs) 1566(1)-(N) residing within the untrusted app subnet(s) 1562. Each tenant VM 1566(1)-(N) can run code in a respective container 1567(1)-(N), and be communicatively coupled to an app subnet 1526 that can be contained in a data plane app tier 1546 that can be contained in a container egress VCN 1568. Respective secondary VNICs 1572(1)-(N) can facilitate communication between the untrusted app subnet(s) 1562 contained in the data plane VCN 1518 and the app subnet contained in the container egress VCN 1568. The container egress VCN can include a NAT gateway 1538 that can be communicatively coupled to public Internet 1554 (e.g., public Internet 1254 of FIG. 12).

The Internet gateway 1534 contained in the control plane VCN 1516 and contained in the data plane VCN 1518 can be communicatively coupled to a metadata management service 1552 (e.g., the metadata management system 1252 of FIG. 12) that can be communicatively coupled to public Internet 1554. Public Internet 1554 can be communicatively coupled to the NAT gateway 1538 contained in the control plane VCN 1516 and contained in the data plane VCN 1518. The service gateway 1536 contained in the control plane VCN 1516 and contained in the data plane VCN 1518 can be communicatively coupled to cloud services 1556.

In some examples, the pattern illustrated by the architecture of block diagram 1500 of FIG. 15 may be considered an exception to the pattern illustrated by the architecture of block diagram 1400 of FIG. 14 and may be desirable for a customer of the IaaS provider if the IaaS provider cannot directly communicate with the customer (e.g., a disconnected region). The respective containers 1567(1)-(N) that are contained in the VMs 1566(1)-(N) for each customer can be accessed in real-time by the customer. The containers 1567(1)-(N) may be configured to make calls to respective secondary VNICs 1572(1)-(N) contained in app subnet(s) 1526 of the data plane app tier 1546 that can be contained in the container egress VCN 1568. The secondary VNICs 1572(1)-(N) can transmit the calls to the NAT gateway 1538 that may transmit the calls to public Internet 1554. In this example, the containers 1567(1)-(N) that can be accessed in real-time by the customer can be isolated from the control plane VCN 1516 and can be isolated from other entities contained in the data plane VCN 1518. The containers 1567(1)-(N) may also be isolated from resources from other customers.

In other examples, the customer can use the containers 1567(1)-(N) to call cloud services 1556. In this example, the customer may run code in the containers 1567(1)-(N) that requests a service from cloud services 1556. The containers 1567(1)-(N) can transmit this request to the secondary VNICs 1572(1)-(N) that can transmit the request to the NAT gateway that can transmit the request to public Internet 1554. Public Internet 1554 can transmit the request to LB subnet(s) 1522 contained in the control plane VCN 1516 via the Internet gateway 1534. In response to determining the request is valid, the LB subnet(s) can transmit the request to app subnet(s) 1526 that can transmit the request to cloud services 1556 via the service gateway 1536.

It should be appreciated that IaaS architectures 1200, 1300, 1400, 1500 depicted in the figures may have other components than those depicted. Further, the embodiments shown in the figures are only some examples of a cloud infrastructure system that may incorporate an embodiment of the disclosure. In some other embodiments, the IaaS systems may have more or fewer components than shown in the figures, may combine two or more components, or may have a different configuration or arrangement of components.

In certain embodiments, the IaaS systems described herein may include a suite of applications, middleware, and database service offerings that are delivered to a customer in a self-service, subscription-based, elastically scalable, reliable, highly available, and secure manner. An example of such an IaaS system is the Oracle Cloud Infrastructure (OCI) provided by the present assignee.

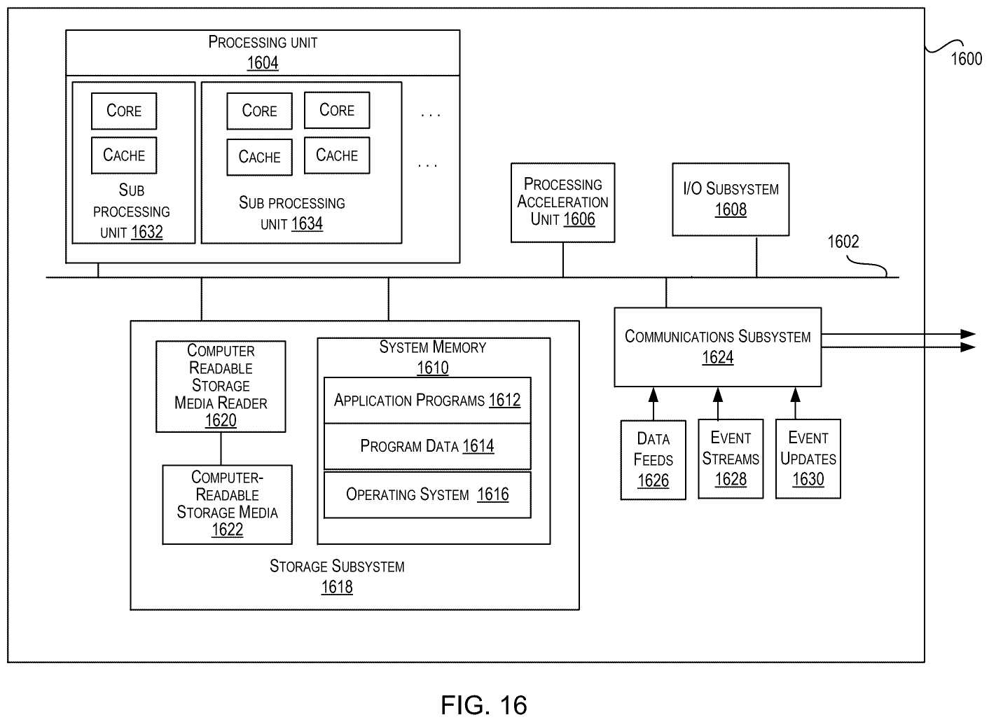

FIG. 16 illustrates an example computer system 1600, in which various embodiments may be implemented. The system 1600 may be used to implement any of the computer systems described above. As shown in the figure, computer system 1600 includes a processing unit 1604 that communicates with a number of peripheral subsystems via a bus subsystem 1602. These peripheral subsystems may include a processing acceleration unit 1606, an I/O subsystem 1608, a storage subsystem 1618 and a communications subsystem 1624. Storage subsystem 1618 includes tangible computer-readable storage media 1622 and a system memory 1610.

Bus subsystem 1602 provides a mechanism for letting the various components and subsystems of computer system 1600 communicate with each other as intended. Although bus subsystem 1602 is shown schematically as a single bus, alternative embodiments of the bus subsystem may utilize multiple buses. Bus subsystem 1602 may be any of several types of bus structures including a memory bus or memory controller, a peripheral bus, and a local bus using any of a variety of bus architectures. For example, such architectures may include an Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus, which can be implemented as a Mezzanine bus manufactured to the IEEE P1386.1 standard.

Processing unit 1604, which can be implemented as one or more integrated circuits (e.g., a conventional microprocessor or microcontroller), controls the operation of computer system 1600. One or more processors may be included in processing unit 1604. These processors may include single core or multicore processors. In certain embodiments, processing unit 1604 may be implemented as one or more independent processing units 1632 and/or 1634 with single or multicore processors included in each processing unit. In other embodiments, processing unit 1604 may also be implemented as a quad-core processing unit formed by integrating two dual-core processors into a single chip.

In various embodiments, processing unit 1604 can execute a variety of programs in response to program code and can maintain multiple concurrently executing programs or processes. At any given time, some or all of the program code to be executed can be resident in processor(s) 1604 and/or in storage subsystem 1618. Through suitable programming, processor(s) 1604 can provide various functionalities described above. Computer system 1600 may additionally include a processing acceleration unit 1606, which can include a digital signal processor (DSP), a special-purpose processor, and/or the like.

I/O subsystem 1608 may include user interface input devices and user interface output devices. User interface input devices may include a keyboard, pointing devices such as a mouse or trackball, a touchpad or touch screen incorporated into a display, a scroll wheel, a click wheel, a dial, a button, a switch, a keypad, audio input devices with voice command recognition systems, microphones, and other types of input devices. User interface input devices may include, for example, motion sensing and/or gesture recognition devices such as the Microsoft Kinect® motion sensor that enables users to control and interact with an input device, such as the Microsoft Xbox® 360 game controller, through a natural user interface using gestures and spoken commands. User interface input devices may also include eye gesture recognition devices such as the Google Glass® blink detector that detects eye activity (e.g., ‘blinking’ while taking pictures and/or making a menu selection) from users and transforms the eye gestures as input into an input device (e.g., Google Glass®). Additionally, user interface input devices may include voice recognition sensing devices that enable users to interact with voice recognition systems (e.g., Siri® navigator), through voice commands.

User interface input devices may also include, without limitation, three dimensional (3D) mice, joysticks or pointing sticks, gamepads and graphic tablets, and audio/visual devices such as speakers, digital cameras, digital camcorders, portable media players, webcams, image scanners, fingerprint scanners, barcode reader 3D scanners, 3D printers, laser rangefinders, and eye gaze tracking devices. Additionally, user interface input devices may include, for example, medical imaging input devices such as computed tomography, magnetic resonance imaging, position emission tomography, medical ultrasonography devices. User interface input devices may also include, for example, audio input devices such as MIDI keyboards, digital musical instruments and the like.

User interface output devices may include a display subsystem, indicator lights, or non-visual displays such as audio output devices, etc. The display subsystem may be a cathode ray tube (CRT), a flat-panel device, such as that using a liquid crystal display (LCD) or plasma display, a projection device, a touch screen, and the like. In general, use of the term “output device” is intended to include all possible types of devices and mechanisms for outputting information from computer system 1600 to a user or other computer. For example, user interface output devices may include, without limitation, a variety of display devices that visually convey text, graphics and audio/video information such as monitors, printers, speakers, headphones, automotive navigation systems, plotters, voice output devices, and modems.

Computer system 1600 may comprise a storage subsystem 1618 that provides a tangible non-transitory computer-readable storage medium for storing software and data constructs that provide the functionality of the embodiments described in this disclosure. The software can include programs, code modules, instructions, scripts, etc., that when executed by one or more cores or processors of processing unit 1604 provide the functionality described above. Storage subsystem 1618 may also provide a repository for storing data used in accordance with the present disclosure.

As depicted in the example in FIG. 16, storage subsystem 1618 can include various components including a system memory 1610, computer-readable storage media 1622, and a computer readable storage media reader 1620. System memory 1610 may store program instructions that are loadable and executable by processing unit 1604. System memory 1610 may also store data that is used during the execution of the instructions and/or data that is generated during the execution of the program instructions. Various different kinds of programs may be loaded into system memory 1610 including but not limited to client applications, Web browsers, mid-tier applications, relational database management systems (RDBMS), virtual machines, containers, etc.

System memory 1610 may also store an operating system 1616. Examples of operating system 1616 may include various versions of Microsoft Windows®, Apple Macintosh®, and/or Linux operating systems, a variety of commercially-available UNIX® or UNIX-like operating systems (including without limitation the variety of GNU/Linux operating systems, the Google Chrome® OS, and the like) and/or mobile operating systems such as iOS, Windows® Phone, Android® OS, BlackBerry® OS, and Palm® OS operating systems. In certain implementations where computer system 1600 executes one or more virtual machines, the virtual machines along with their guest operating systems (GOSs) may be loaded into system memory 1610 and executed by one or more processors or cores of processing unit 1604.

System memory 1610 can come in different configurations depending upon the type of computer system 1600. For example, system memory 1610 may be volatile memory (such as random access memory (RAM)) and/or non-volatile memory (such as read-only memory (ROM), flash memory, etc.) Different types of RAM configurations may be provided including a static random access memory (SRAM), a dynamic random access memory (DRAM), and others. In some implementations, system memory 1610 may include a basic input/output system (BIOS) containing basic routines that help to transfer information between elements within computer system 1600, such as during start-up.

Computer-readable storage media 1622 may represent remote, local, fixed, and/or removable storage devices plus storage media for temporarily and/or more permanently containing, storing, computer-readable information for use by computer system 1600 including instructions executable by processing unit 1604 of computer system 1600.

Computer-readable storage media 1622 can include any appropriate media known or used in the art, including storage media and communication media, such as but not limited to, volatile and non-volatile, removable and non-removable media implemented in any method or technology for storage and/or transmission of information. This can include tangible computer-readable storage media such as RAM, ROM, electronically erasable programmable ROM (EEPROM), flash memory or other memory technology, CD-ROM, digital versatile disk (DVD), or other optical storage, magnetic cassettes, magnetic tape, magnetic disk storage or other magnetic storage devices, or other tangible computer readable media.

By way of example, computer-readable storage media 1622 may include a hard disk drive that reads from or writes to non-removable, nonvolatile magnetic media, a magnetic disk drive that reads from or writes to a removable, nonvolatile magnetic disk, and an optical disk drive that reads from or writes to a removable, nonvolatile optical disk such as a CD ROM, DVD, and Blu-Ray® disk, or other optical media. Computer-readable storage media 1622 may include, but is not limited to, Zip® drives, flash memory cards, universal serial bus (USB) flash drives, secure digital (SD) cards, DVD disks, digital video tape, and the like. Computer-readable storage media 1622 may also include, solid-state drives (SSD) based on non-volatile memory such as flash-memory based SSDs, enterprise flash drives, solid state ROM, and the like, SSDs based on volatile memory such as solid state RAM, dynamic RAM, static RAM, DRAM-based SSDs, magnetoresistive RAM (MRAM) SSDs, and hybrid SSDs that use a combination of DRAM and flash memory based SSDs. The disk drives and their associated computer-readable media may provide non-volatile storage of computer-readable instructions, data structures, program modules, and other data for computer system 1600.

Machine-readable instructions executable by one or more processors or cores of processing unit 1604 may be stored on a non-transitory computer-readable storage medium. A non-transitory computer-readable storage medium can include physically tangible memory or storage devices that include volatile memory storage devices and/or non-volatile storage devices. Examples of non-transitory computer-readable storage medium include magnetic storage media (e.g., disk or tapes), optical storage media (e.g., DVDs, CDs), various types of RAM, ROM, or flash memory, hard drives, floppy drives, detachable memory drives (e.g., USB drives), or other type of storage device.

Communications subsystem 1624 provides an interface to other computer systems and networks. Communications subsystem 1624 serves as an interface for receiving data from and transmitting data to other systems from computer system 1600. For example, communications subsystem 1624 may enable computer system 1600 to connect to one or more devices via the Internet. In some embodiments communications subsystem 1624 can include radio frequency (RF) transceiver components for accessing wireless voice and/or data networks (e.g., using cellular telephone technology, advanced data network technology, such as 3G, 4G or EDGE (enhanced data rates for global evolution), WiFi (IEEE 802.11 family standards, or other mobile communication technologies, or any combination thereof), global positioning system (GPS) receiver components, and/or other components. In some embodiments communications subsystem 1624 can provide wired network connectivity (e.g., Ethernet) in addition to or instead of a wireless interface.

In some embodiments, communications subsystem 1624 may also receive input communication in the form of structured and/or unstructured data feeds 1626, event streams 1628, event updates 1630, and the like on behalf of one or more users who may use computer system 1600.

By way of example, communications subsystem 1624 may be configured to receive data feeds 1626 in real-time from users of social networks and/or other communication services such as Twitter® feeds, Facebook® updates, web feeds such as Rich Site Summary (RSS) feeds, and/or real-time updates from one or more third party information sources.

Additionally, communications subsystem 1624 may also be configured to receive data in the form of continuous data streams, which may include event streams 1628 of real-time events and/or event updates 1630, that may be continuous or unbounded in nature with no explicit end. Examples of applications that generate continuous data may include, for example, sensor data applications, financial tickers, network performance measuring tools (e.g., network monitoring and traffic management applications), clickstream analysis tools, automobile traffic monitoring, and the like.

Communications subsystem 1624 may also be configured to output the structured and/or unstructured data feeds 1626, event streams 1628, event updates 1630, and the like to one or more databases that may be in communication with one or more streaming data source computers coupled to computer system 1600.

Computer system 1600 can be one of various types, including a handheld portable device (e.g., an iPhone® cellular phone, an iPad® computing tablet, a PDA), a wearable device (e.g., a Google Glass® head mounted display), a PC, a workstation, a mainframe, a kiosk, a server rack, or any other data processing system.

Due to the ever-changing nature of computers and networks, the description of computer system 1600 depicted in the figure is intended only as a specific example. Many other configurations having more or fewer components than the system depicted in the figure are possible. For example, customized hardware might also be used and/or particular elements might be implemented in hardware, firmware, software (including applets), or a combination. Further, connection to other computing devices, such as network input/output devices, may be employed. Based on the disclosure and teachings provided herein, a person of ordinary skill in the art will appreciate other ways and/or methods to implement the various embodiments.

Although specific embodiments have been described, various modifications, alterations, alternative constructions, and equivalents are also encompassed within the scope of the disclosure. Embodiments are not restricted to operation within certain specific data processing environments, but are free to operate within a plurality of data processing environments. Additionally, although embodiments have been described using a particular series of transactions and steps, it should be apparent to those skilled in the art that the scope of the present disclosure is not limited to the described series of transactions and steps. Various features and aspects of the above-described embodiments may be used individually or jointly.

Further, while embodiments have been described using a particular combination of hardware and software, it should be recognized that other combinations of hardware and software are also within the scope of the present disclosure. Embodiments may be implemented only in hardware, or only in software, or using combinations thereof. The various processes described herein can be implemented on the same processor or different processors in any combination. Accordingly, where components or services are described as being configured to perform certain operations, such configuration can be accomplished, e.g., by designing electronic circuits to perform the operation, by programming programmable electronic circuits (such as microprocessors) to perform the operation, or any combination thereof. Processes can communicate using a variety of techniques including but not limited to conventional techniques for inter process communication, and different pairs of processes may use different techniques, or the same pair of processes may use different techniques at different times.

The specification and drawings are, accordingly, to be regarded in an illustrative rather than a restrictive sense. It will, however, be evident that additions, subtractions, deletions, and other modifications and changes may be made thereunto without departing from the broader spirit and scope as set forth in the claims. Thus, although specific disclosure embodiments have been described, these are not intended to be limiting. Various modifications and equivalents are within the scope of the following claims.

The use of the terms “a” and “an” and “the” and similar referents in the context of describing the disclosed embodiments (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments and does not pose a limitation on the scope of the disclosure unless otherwise claimed. No language in the specification should be construed as indicating any non-claimed element as essential to the practice of the disclosure.

Disjunctive language such as the phrase “at least one of X, Y, or Z,” unless specifically stated otherwise, is intended to be understood within the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

Preferred embodiments of this disclosure are described herein, including the best mode known for carrying out the disclosure. Variations of those preferred embodiments may become apparent to those of ordinary skill in the art upon reading the foregoing description. Those of ordinary skill should be able to employ such variations as appropriate and the disclosure may be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein.

All references, including publications, patent applications, and patents, cited herein are hereby incorporated by reference to the same extent as if each reference were individually and specifically indicated to be incorporated by reference and were set forth in its entirety herein.

In the foregoing specification, aspects of the disclosure are described with reference to specific embodiments thereof, but those skilled in the art will recognize that the disclosure is not limited thereto. Various features and aspects of the above-described disclosure may be used individually or jointly. Further, embodiments can be utilized in any number of environments and applications beyond those described herein without departing from the broader spirit and scope of the specification. The specification and drawings are, accordingly, to be regarded as illustrative rather than restrictive.

What is claimed is:

1. A computer-implemented method comprising:
obtaining an original dataset for training a machine learning model to generate a synthetic doctor-patient conversation, the original dataset including data in the form of textual dialogue associated with each of a plurality of individual doctor-patient conversations;
constructing input data by performing a named entity recognition operation on the dialogue of the original dataset that captures and categorizes named medical entities present in the dialogue;
generating prepared input data by arranging the input data in an annotated turn-by-turn conversation format using an input data preparation algorithm having various control parameters;
training the machine learning model using the prepared input data;
randomly selecting a disease from a medical knowledge graph and capturing a plurality of symptoms that are mapped to the selected disease; and
causing the trained machine learning model to generate a turn-by-turn synthetic doctor-patient conversation according to a conversation generation control algorithm having various control parameters, by inputting the plurality of symptoms to the machine learning model as a first control parameter of the conversation generation control algorithm and randomly assigning a first speaker.

2. The computer-implemented method of claim 1, wherein the named medical entities are selected from the group consisting of biomedical information, personal identifying information, personal health information, and combinations thereof.

3. The computer-implemented method of claim 1, wherein constructing the input data further comprises:
post-processing the named medical entities to remove any tagging anomalies resulting from the named entity recognition operation; and
performing an additional medical named entity recognition operation on the post-processed named medical entities using at least one model trained on a biomedical corpus.

4. The computer-implemented method of claim 1, wherein:
the control parameters of the input data preparation algorithm and the conversation generation control algorithm include symptoms/topics, entities sampled from the symptoms/topics, speaker identity, conversation turn length, remaining conversation turns, and context; and
wherein a value of the context control parameter for a given conversation turn comprises the cumulative dialogue of all the preceding conversation turns, when there is at least one preceding conversation turn.

5. The computer-implemented method of claim 4, wherein generating the prepared input data further comprises:
comparing the total token length of the context parameter value to a predetermined maximum allowable context token length value; and
when the total token length of the context parameter value exceeds the predetermined maximum allowable context token length value, remove dialogue turns one at a time from the context parameter until the total token length value of the context parameter no longer exceeds the maximum allowable context token length value.

6. The computer-implemented method of claim 4, wherein training the machine learning model includes multistage finetuning of the machine learning model, the multistage finetuning comprising:
first-fold finetuning using a medical question answering dataset;
following first-fold finetuning, second-fold finetuning guided by the input data preparation algorithm and the control parameters thereof and using a dataset including data in the form of textual dialogue associated with each of a plurality of individual non-medical specific conversations; and
following second-fold finetuning, final-fold finetuning guided by the input data preparation algorithm and the control parameters thereof and using a dataset including data in the form of textual dialogue associated with each of a plurality of individual medical-specific conversations, wherein the symptoms/topics control parameter of the input data preparation algorithm is composed of keywords previously derived from a dataset including data in the form of textual dialogue associated with each of a plurality of individual non-medical conversations.

7. The computer-implemented method of claim 1, further comprising evaluating performance of the trained machine learning model by analyzing the generated turn-by-turn synthetic doctor-patient conversation using a metric selected from the group consisting of recall-oriented understudy for gisting evaluation, n-gram diversity score, unique n-gram count, and combinations thereof.

8. A system comprising:
one or more data processors; and
one or more non-transitory computer readable media storing instructions which, when executed by the one or more data processors, cause the one or more data processors to perform the following operations:
obtaining an original dataset for training a machine learning model to generate a synthetic doctor-patient conversation, the original dataset including data in the form of textual dialogue associated with each of a plurality of individual doctor-patient conversations;
constructing input data by performing a named entity recognition operation on the dialogue of the original dataset that captures and categorizes named medical entities present in the dialogue;
generating prepared input data by arranging the input data in an annotated turn-by-turn conversation format using an input data preparation algorithm having various control parameters;
training the machine learning model using the prepared input data;
randomly selecting a disease from a medical knowledge graph and capturing a plurality of symptoms that are mapped to the selected disease; and
causing the trained machine learning model to generate a turn-by-turn synthetic doctor-patient conversation according to a conversation generation control algorithm having various control parameters, by inputting the plurality of symptoms to the machine learning model as a first control parameter of the conversation generation control algorithm and randomly assigning a first speaker.

9. The system of claim 8, wherein the named medical entities are selected from the group consisting of biomedical information, personal identifying information, personal health information, and combinations thereof.

10. The system of claim 8, wherein:
the control parameters of the input data preparation algorithm and the conversation generation control algorithm include symptoms/topics, entities sampled from the symptoms/topics, speaker identity, conversation turn length, remaining conversation turns, and context; and
wherein a value of the context control parameter for a given conversation turn comprises the dialogue of all preceding conversation turns, when there is at least one preceding conversation turn.

11. The system of claim 10, wherein the format of the prepared input data is:
[TOPICS] <symptom values> [ENTITIES] <entity values> [SPEAKER]
<doctor/patient> [TURN_LENGTH]
<length of the turn>[REMAINING_TURNS]
<number of turns left> [CONTEXT] <previous dialogues if available>.

12. The system of claim 10, wherein training the machine learning model includes multistage finetuning of the machine learning model, the multistage finetuning comprising:
first-fold finetuning using a medical question answering dataset;
following first-fold finetuning, second-fold finetuning guided by the input data preparation algorithm and the control parameters thereof and using a dataset including data in the form of textual dialogue associated with each of a plurality of individual non-medical specific conversations; and
following second-fold finetuning, final-fold finetuning guided by the input data preparation algorithm and the control parameters thereof and using a dataset including data in the form of textual dialogue associated with each of a plurality of individual medical-specific conversations, wherein the symptoms/topics control parameter of the input data preparation algorithm is composed of keywords previously derived from a dataset including data in the form of textual dialogue associated with each of a plurality of individual non-medical conversations.

13. The system of claim 8, wherein performance of the trained machine learning model is evaluatable by analyzing the generated turn-by-turn synthetic doctor-patient conversation using a metric selected from the group consisting of recall-oriented understudy for gisting evaluation, n-gram diversity score, unique n-gram count, and combinations thereof.

14. The system of claim 8, wherein the machine learning model has a transformer-encoder-decoder architecture.

15. A computer-program product tangibly embodied in one or more non-transitory machine-readable media, including instructions configured to cause one or more data processors to perform the following operations:
obtaining an original dataset for training a machine learning model to generate a synthetic doctor-patient conversation, the original dataset including data in the form of textual dialogue associated with each of a plurality of individual doctor-patient conversations;
constructing input data by performing a named entity recognition operation on the dialogue of the original dataset that captures and categorizes named medical entities present in the dialogue;
generating prepared input data by arranging the input data in an annotated turn-by-turn conversation format using an input data preparation algorithm having various control parameters;
training the machine learning model using the prepared input data;
randomly selecting a disease from a medical knowledge graph and capturing a plurality of symptoms that are mapped to the selected disease; and
causing the trained machine learning model to generate a turn-by-turn synthetic doctor-patient conversation according to a conversation generation control algorithm having various control parameters, by inputting the plurality of symptoms to the machine learning model as a first control parameter of the conversation generation control algorithm and randomly assigning a first speaker.

16. The computer-program product of claim 15, wherein the named medical entities are selected from the group consisting of biomedical information, personal identifying information, personal health information, and combinations thereof.

17. The computer-program product of claim 15, wherein:
the control parameters of the input data preparation algorithm and the conversation generation control algorithm include symptoms/topics, entities sampled from the symptoms/topics, speaker identity, conversation turn length, remaining conversation turns, and context; and
wherein a value of the context control parameter for a given conversation turn comprises the dialogue of all preceding conversation turns, when there is at least one preceding conversation turn.

18. The computer-program product of claim 17, wherein the format of the prepared input data is:
[TOPICS] <symptom values> [ENTITIES] <entity values> [SPEAKER]
<doctor/patient> [TURN_LENGTH]
<length of the turn>[REMAINING_TURNS]
<number of turns left> [CONTEXT] <previous dialogues if available>.

19. The computer-program product of claim 17, wherein training the machine learning model includes multistage finetuning of the machine learning model, the multistage finetuning comprising:
first-fold finetuning using a medical question answering dataset;
following first-fold finetuning, second-fold finetuning guided by the input data preparation algorithm and the control parameters thereof and using a dataset including data in the form of textual dialogue associated with each of a plurality of individual non-medical specific conversations; and
following second-fold finetuning, final-fold finetuning guided by the input data preparation algorithm and the control parameters thereof and using a dataset including data in the form of textual dialogue associated with each of a plurality of individual medical-specific conversations, wherein the symptoms/topics control parameter of the input data preparation algorithm is composed of keywords previously derived from a dataset including data in the form of textual dialogue associated with each of a plurality of individual non-medical conversations.

20. The computer-program product of claim 15, wherein the machine learning model has a transformer-encoder-decoder architecture.
